(19) United States
(12) Patent Application Publication
(10) Pub. No.: US 2024/0271185 A1
(43) Pub. Date: Aug. 15, 2024

(54) METHODS AND COMPOSITIONS FOR IDENTIFYING METHYLATED CYTOSINES

(71) Applicant: Illumina, Inc., San Diego, CA (US)

(72) Inventors: Colin Brown, San Diego, CA (US); Xiaohai Liu, Cambridge (GB); Xiaolin Wu, Cambridge (GB); Eric Brustad, San Diego, CA (US); Sarah E. Shultzaberger, San Diego, CA (US)

(21) Appl. No.: 18/569,192

(22) PCT Filed: Aug. 16, 2022
(86) PCT No.: PCT/US2022/074999

§ 371 Date: Dec. 11, 2023

Related U.S. Application Data
(60) Provisional application No. 63/234,183, filed on Aug. 17, 2021.

(51) Int. Cl. C12Q 1/6806; C12Q 1/26; C12Q 1/6869

(52) U.S. Cl. CPC C12Q 1/6806; C12Q 1/26; C12Q 1/6869

(57) ABSTRACT

Disclosed herein include methods, compositions, reaction mixtures, kits and systems for identification of methylated cytosines in nucleic acids using a bisulfite-free, one-step chemoenzymatic modification of methylated cytosines.

### CROSS-REFERENCE TO RELATED APPLICATIONS

This application claims the benefit under 35 U.S.C. § 119(e) to U.S. Provisional Patent Application No. 63/234,183 filed on Aug. 17, 2021, the content of which is incorporated herein by reference in its entirety for all purposes.

### REFERENCE TO SEQUENCE LISTING

The present application is being filed along with a Sequence Listing in electronic format. The Sequence Listing is provided as a file entitled 47CX-311977-WO, created Jun. 29, 2022, which is 18.5 kilobytes in size. The information in the electronic format of the Sequence Listing is incorporated herein by reference in its entirety.

### BACKGROUND

### Field

The present disclosure relates generally to the field of molecular biology, for example nucleic acid sequence analysis.

### Description of the Related Art

Detection of methyl cytosine (MeC) is of high interest and importance for understanding epigenetic markers that are implicated in many diseases, including cancer and diabetes. A number of sequencing strategies have been developed to detect methyl cytosine (MeC) and hydroxymethyl cytosine (HO-MeC) on sequencing platforms. These methods involve varying strategies to modify cytosine or methylcytosine adducts during library preparation.

Current methods for detecting nucleic acid methylation and hydroxymethylation often involve multistep processes that require multiple enzymatic modifications and/or chemical modifications of cytosine or methylcytosine and require complicated workflows. For example, some of these methods employ bisulfite treatment to convert unmethylated cytosine to uracil while leaving 5-methylcytosine (5mC) and/or 5-hydroxymethylcytosine (5hmC) intact. Also available are enzymatic methyl-seq (EM-Seq) methods which employ oxygenase and cytosine deaminase to convert unmethylated cytosine to uracil while leaving 5mC and/or 5hmC intact, and Tet-assisted pyridine borane sequencing (TAPS) methods which employ oxygenase and borane reagent to convert methylated cytosine to dihydrouracil.

There are however several drawbacks to these methods. First, bisulfite treatment is a harsh chemical reaction, which degrades more than 90% of the DNA due to depurination under the required acidic and thermal conditions. This degradation severely limits its application to low-input samples. Second, both bisulfite sequencing and EM-seq rely on the complete conversion of unmodified cytosine to thymine. Unmodified cytosine accounts for approximately 95% of the total cytosine in the human genome. Converting all these positions to thymine severely reduces sequence complexity, leading to poor sequencing quality, low mapping rates, uneven genome coverage and increased sequencing cost. Third, both EM-Seq and TAPS employ a two-step chemical modification, which are susceptible to false detection of 5mC and 5hmC due to incomplete conversion of methylated cytosine to 5-carboxy cytosine. Fourth, the borane reductant used in TAPS is also potentially toxic.

There is a need for a method for nucleic acid methylation and hydroxymethylation analysis that is a mild, nontoxic reaction, can detect the methylated cytosine (5mC and/or 5hmC) at base resolution without affecting the unmethylated cytosine, and uses a one-step chemoenzymatic reaction to simply the process.

### SUMMARY

Disclosed herein include methods and reaction mixtures for identifying 5-methylcytosine (5mC), 5-hydroxymethylcytosine (5hmC), or both in a target nucleic acid. The method can comprise providing a nucleic acid sample comprising a target nucleic acid suspected of comprising, or comprising, one or more 5-methylcytosine (5mC) or 5-hydroxymethylcytosine (5hmC), performing a ten eleven translocation enzyme (TET)-mediated carbene insertion on the 5-methyl moiety of the 5mC or the 5-hydroxymethyl moiety of 5hmC in the target nucleic acid to generate a modified target nucleic acid, and determining the sequence of the modified target nucleic acid, wherein a cytosine (C) to (T) transition in the sequence of the modified target nucleic acid compared to the sequence of the target nucleic acid indicates a 5mC or 5hmC in the target nucleic acid.

In some embodiments, the method comprises contacting the target nucleic acid with a TET or a variant thereof, thereby producing a C—H insertion on the 5-methyl moiety of the 5mC or the 5-hydroxymethyl moiety of 5hmC. In some embodiments, the TET-mediated carbene insertion comprises converting the 5mC or 5hmC into a modified nucleic acid adduct capable of forming a hydrogen bond with adenine (A). In some embodiments, the TET-mediated carbene insertion is performed in the presence of a carbene precursor. In some embodiments, the method can comprise amplifying the modified target nucleic acid after (b) and before (c). In some embodiments, the method disclosed herein can comprise performing a TET-mediated carbene insertion on the 5-methyl moiety of the 5mC or the 5-hydroxymethyl moiety of 5hmC under an anaerobic condition. In some embodiments, the method disclosed herein can comprise performing a TET-mediated carbene insertion on the 5-methyl moiety of the 5mC or the 5-hydroxymethyl moiety of 5hmC under an aerobic condition. In some embodiments, the method disclosed herein can comprise performing a TET-mediated carbene insertion on the 5-methyl moiety of the 5mC or the 5-hydroxymethyl moiety of 5hmC in the presence of a non-reducing acid or a salt thereof.

In some embodiments, the method does not comprise formation of one or more of carboxy cytosine, 5-formyl cytosine, dihydrouracil and uracil. In some embodiments, the method does not comprise conversion of 5mC to carboxy cytosine. In some embodiments, the method does not comprise a deamination reaction by a cytidine deaminase (for example, an APOBEC. (“apolipoprotein B mRNA editing enzyme, catalytic polypeptide-like”). In some embodiments, the method does not comprise chemical reduction by a borane reagent. In some embodiments, the method does not comprise the use of a borane reagent.

Also disclosed herein include a reaction mixture for performing a ten eleven translocation enzyme (TET)-mediated carbene insertion in a nucleic acid comprising 5-methylcytosine (5mC), 5-hydroxymethylcytosine (5hmC) or both. The reaction mixture can comprise a nucleic acid comprising one or more 5-methylcytosine (5mC) or 5-hydroxymethylcytosine (5hmC), a carbene precursor herein disclosed for producing a C—H insertion in the 5-methyl moiety of 5mC or the 5-hydroxymethyl moiety of 5hmC, and a TET or a variant thereof as described herein. In some embodiments, the nucleic acid comprises 5-methylcytosine (5mC), 5-hydroxymethylcytosine (5hmC) or both. In some embodiments, the nucleic acid is suspected of comprising 5-methylcytosine (5mC), 5-hydroxymethylcytosine (5hmC) or both. In some embodiments, the reaction mixture is for a reaction under an anaerobic condition. In some embodiments, the reaction mixture can comprise a non-reducing acid or a salt thereof. The reaction mixture, in some embodiments, does not comprise carboxy cytosine, dihydrouracil, uracil, or a combination thereof. In some embodiments, reaction mixture does not comprise a cytidine deaminase, for example an APOBEC. In some embodiments, the reaction mixture does not comprise a borane reagent.

In some embodiments, the carbene precursor has a structure of Formula I.

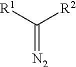

wherein
R¹ is selected from the group consisting of H, —C(O)OR^{1a}, —C(O)R^{1a}, —C(O)N(R^{1b})₂, —SO₂Ria, —SO₂OR¹, —P(O)(OR^{1a})₂, —NO₂, —CN, C₁₋₁₈ alkyl, C₂₋₁₈ alkenyl, C₂₋₁₈ alkynyl, 2- to 18-membered heteroalkyl, C₁₋₁₈ haloalkyl, C₁₋₁₈ alkoxy, C₃₋₁₀cycloalkyl, C₆₋₁₀ aryl, 3- to 10-membered heterocyclyl, and 5- to 10-membered heteroaryl;
each R^{1a} is independently selected from the group consisting of H, C₁₋₁₈ alkyl, C₂₋₁₈ alkenyl, C₂₋₁₈ alkynyl, C₆₋₁₀ aryl, 3- to 10-membered heterocyclyl, and 5- to 10-membered heteroaryl;
each R^{1b} is independently selected from the group consisting of H, C₁₋₁₈ alkyl, C₂₋₁₈ alkenyl, C-₁₈ alkynyl, and C₁₋₁₈ alkoxy;
R² is an electron-withdrawing group selected from the group consisting of —C(O)OR^{2a}, —C(O)R^{2a}, —C(O)N(R^{2b})₂, —SO₂R^{2a}, —SO₂OR^{2a}, —P(O)(OR^{2a})₂, —NO₂, and —CN;
each R^{2a} is independently selected from the group consisting of H, C₁₋₁₈ alkyl, C₂₋₁₈ alkenyl, C₂₋₁₈ alkynyl, C₆₋₁₀ aryl, 3- to 10-membered heterocyclyl, and 5- to 10-membered heteroaryl;
each R^{2b} is independently selected from the group consisting of H, C₁₋₁₈ alkyl, C₂₋₁₈ alkenyl, C₂₋₁₈ alkynyl, and C₁₋₈ alkoxy; and
R¹ and R² are optionally and independently substituted; or
R¹ and R² are taken together to form C₃₋₁₀ cycloalkyl, C₆₋₁₀ aryl, 3- to 10-membered heterocyclyl, and 5- to 10-membered heteroaryl, each of which is optionally substituted.

In some embodiments, the carbene precursor is a compound according to Formula I wherein
R¹ is selected from the group consisting of H, —C(O)OR^{1a}, —C(O)R^{1a}, C(O)N(R^{1b})₂, —SO₂Ria, —SO₂OR^{1a}, —P(O)(OR^{1a})₂, —NO₂, —CN, C₁₋₁₈ alkyl, 2- to 18-membered heteroalkyl, C₁₋₁₈haloalkyl, C₁₋₁₈alkoxy, C₃₋₁₀cycloalkyl, C₆₋₁₀aryl, 3- to 10-membered heterocyclyl, and 5- to 10-membered heteroaryl;
each R^{1a} is independently C₁₋₈ alkyl;
each R^{1b} is independently selected from the group consisting of H, C₁₋₈ alkyl, and C₁₋₈ alkoxy;
R² is an electron-withdrawing group selected from the group consisting of —C(O)OR^{2a}, —C(O)R^{2a}, —C(O)N(R^{2b})₂, —SO₂R^{2a}, —SO₂OR^{2a}, —P(O)(OR^{2a})₂, —NO₂, and —CN;
each R^{2a} is independently C₁₋₈ alkyl;
each R^{2b} is independently selected from the group consisting of H, C₁₋₈ alkyl, and C₁₋₈ alkoxy; and
R¹ and R² are optionally and independently substituted; or
R¹ and R² are taken together to form C₃₋₁₀ cycloalkyl, C₆₋₁₀ aryl, 3- to 10-membered heterocyclyl, and 5- to 10-membered heteroaryl, each of which is optionally substituted.

In some embodiments, the carbene precursor is a compound according to Formula I wherein
R¹ is independently selected from the group consisting of H, —C(O)OR^{1a}, —C(O)R^{1a}, —SO₂R^{1a}, —SO₂OR^{1a}, substituted C₁₋₁₈ alkyl, 2- to 18-membered heteroalkyl, C₁₋₁₈ alkoxy, C₃₋₁₀ cycloalkyl, C₁₋₁₈ fluoroalkyl, substituted C₆₋₁₀ aryl, and substituted 5- to 10-membered heteroaryl;
R^{1a} is C₁₋₈ alkyl;
R² is selected from the group consisting of —C(O)OR^{2a}, —C(O)R^{2a}, —SO₂R^{2a}, and —SO₂OR^{2a}; and
R^{2a} is C₁₋₈ alkyl; or
R¹ and R² are optionally taken together to form C₃₋₁₀ cycloalkyl, C₆₋₁₀ aryl, 3- to 10-membered heterocyclyl, and 5- to 10-membered heteroaryl, each of which is optionally substituted.

In some embodiments, the carbene precursor is selected from the group consisting of diazo reagents, diazirine reagents, hydrazone reagents, and a combination thereof. In some embodiments, the carbene precursor is selected from the group consisting of:

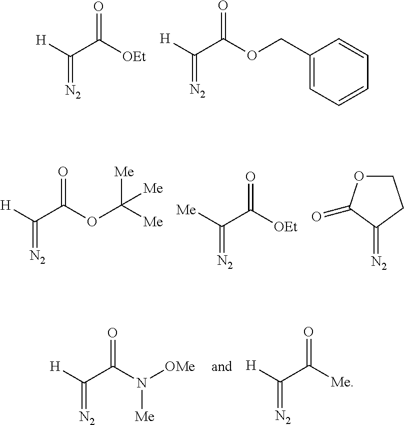

wherein “Me” denotes a methyl group and “Et” denotes an ethyl group.

In some embodiments, the carbene precursor is diazoacetate ester.

In some embodiments, the TET is selected from the group consisting of human TET1, TET2, TET3, and variants thereof; murine Tet1, Tet2, Tet3, and variants thereof; Naegleria TET (NgTET) and variants thereof; Coprinopsis cinerea (CcTET) and variants thereof; and a combination thereof. In some embodiments, the TET is TET1. In some embodiments, the TET is NgTET. In some embodiments, the ten eleven translocation enzyme (TET)-mediated carbene insertion on the 5-methyl moiety of the 5mC or the 5-hydroxymethyl moiety of 5hmC in the target nucleic acid to generate a modified target nucleic acid is carried out by a TET-like enzyme, for example a TET-like dioxygenase.

In some embodiments, a cofactor alpha-ketoglutarate of the TET or a variant thereof is replaced with a non-reducing acid or a salt thereof. The non-reducing acid can be selected from the group consisting of acetic acid, dichloroacetic acid, fluoroacetic acid, chloroacetic acid, citric acid, ascorbic acid, benzoic acid, and a combination thereof. In some embodiments, the non-reducing acid is acetic acid. In some embodiments, the non-reducing acid is a structural analog of alpha-ketoglutarate (aKG), including but not limited to n-oxalylglycine.

In some embodiments, the target nucleic acid comprises at least one 5mC. The target nucleic acid can be DNA or RNA. In some embodiments, the target nucleic acid is mammalian genomic DNA. In some embodiments, the target nucleic acid is human genomic DNA. In some embodiments, the nucleic acid sample is selected from the group consisting of a clinical sample and a derivative thereof, an environmental sample and a derivative thereof, an agricultural sample and a derivative thereof, and a combination thereof.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 illustrates heterogeneous oxidation of MeC via the TET enzyme.

FIG. 2 illustrates a wild type catalysis (monooxygenation), a carbene insertion (C—C bond formation) reaction and a nitrene insertion (C—N bond formation) reaction carried out by heme bound proteins such as cytochrome P450.

FIG. 3 illustrates a wild type catalysis (monooxygenation), a carbene insertion (C—C bond formation) reaction and a nitrene insertion (C—N bond formation) reactions carried out by non-heme iron oxidases such as TET.

FIG. 4 illustrates a non-natural carbene-modification of MeC by TET in comparison to the natural TET-mediate oxidation reaction. The left panel of FIG. 4 shows a crystal structure of the iron-containing active site of TET. The top row of the right panel illustrates a natural TET-mediated oxidation of MeC. The bottom row of the right panel illustrates a modified, non-natural TET-mediated carbene-insertion followed by spontaneous cyclization and tautomerization to generate a novel sequenceable base.

FIG. 5 illustrates the cyclization and tautomerization of the cyclized product following the carbene-insertion in the methyl moiety of a 5-mC in order to alter the Watson-Crick hydrogen bonding face of the modified-MeC base.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for identifying 5-methylcytosine (5mC), 5-hydroxymethylcytosine (5hmC), or both in a target nucleic acid. The methods disclosed herein can perform nucleic acid methylation and hydroxymethylation analysis in a mild, nontoxic reaction and use a bisulfite-free, one-step chemoenzymatic modification of methylated cytosines to simply the reaction. When used in conjunction with sequencing techniques, the methods disclosed herein can detect methylated cytosines (5mC and 5hmC) at base resolution without affecting the unmethylated cytosine. Also provided herein include reaction mixtures for performing a ten eleven translocation enzyme (TET)-mediated carbene insertion in a nucleic acid comprising 5-methylcytosine (5mC), 5-hydroxymethylcytosine (5hmC) or both.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, N Y 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The terms “protein,” “peptide,” and “polypeptide” are used interchangeably herein to refer to a polymer of amino acid residues, or an assembly of multiple polymers of amino acid residues. The terms apply to amino acid polymers in which one or more amino acid residues are an artificial chemical mimic of a corresponding naturally occurring amino acid, as well as to naturally occurring amino acid polymers and non-naturally occurring amino acid polymers.

The term “amino acid” includes naturally-occurring α-amino acids and their stereoisomers, as well as unnatural (non-naturally occurring) amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid, i.e., the D-amino acid.

Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” are unnatural amino acids that have the same basic chemical structure as naturally-occurring amino acids, i.e., an a carbon that is bound to a hydrogen, a carboxyl group, an amino group, but have modified R (i.e., side-chain) groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either their commonly known three letter symbols or by the one-letter symbols recommended by the IUPAC-IUB Biochemical Nomenclature Commission. For example, an L-amino acid may be represented herein by its commonly known three letter symbol (e.g., Arg for L-arginine) or by an upper-case one-letter amino acid symbol (e.g., R for L-arginine). A D-amino acid may be represented herein by its commonly known three letter symbol (e.g., D-Arg for D-arginine) or by a lower-case one-letter amino acid symbol (e.g., r for D-arginine).

As used herein, the term “variant” refers to a polynucleotide or polypeptide having a sequence substantially similar to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

The term “site-directed mutagenesis” refers to various methods in which specific changes are intentionally made introduced into a nucleotide sequence (i.e., specific nucleotide changes are introduced at pre-determined locations). Known methods of performing site-directed mutagenesis include, but are not limited to, PCR site-directed mutagenesis, cassette mutagenesis, whole plasmid mutagenesis, and Kunkel's method.

The term “site-saturation mutagenesis,” also known as “saturation mutagenesis,” refers to a method of introducing random mutations at predetermined locations with a nucleotide sequence, and is a method commonly used in the context of directed evolution (e.g., the optimization of proteins (e.g., in order to enhance activity, stability, and/or stability), metabolic pathways, and genomes). In site-saturation mutagenesis, artificial gene sequences are synthesized using one or more primers that contain degenerate codons; these degenerate codons introduce variability into the position(s) being optimized. Each of the three positions within a degenerate codon encodes a base such as adenine (A), cytosine (C), thymine (T), or guanine (G), or encodes a degenerate position such as K (which can be G or T), M (which can be A or C), R (which can be A or G), S (which can be C or G), W (which can be A or T), Y (which can be C or T), B (which can be C, G, or T), D (which can be A, G, or T), H (which can be A, C, or T), V (which can be A, C, or G), or N (which can be A, C, G, or T). Thus, as a non-limiting example, the degenerate codon NDT encodes an A, C, G, or T at the first position, an A, G, or T at the second position, and a T at the third position. This particular combination of 12 codons represents 12 amino acids (Phe, Leu, Ile, Val, Tyr, His, Asn, Asp, Cys, Arg, Ser, and Gly). As another non-limiting example, the degenerate codon VHG encodes an A, C, or G at the first position, an A, C, or T at the second position, and G at the third position. This particular combination of 9 codons represents 8 amino acids (Lys, Thr, Met, Glu, Pro, Leu, Ala, and Val). As another non-limiting example, the “fully randomized” degenerate codon NNN includes all 64 codons and represents all 20 naturally-occurring amino acids.

The term “DNA methylation” is an epigenetic mechanism that occurs by the addition of a methyl group to cytosine bases within genomic DNA, typically in CpG islands, thereby modifying the function of the genes and affecting gene expression. The most characterized DNA methylation process is the covalent addition of the methyl group at the 5-carbon of the cytosine ring resulting in 5-methycytosine (5-mC). This methyl group can be further modified to hydroxymethyl cytosine (5-hmC) by the addition of a single hydroxyl moiety. The term “methylated cytosine” “MeC” used herein refers to 5-mC, 5-hmC, or both.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can refer to alkyl groups having up to 20 carbons atoms, such as, but not limited to heptyl, octyl, nonyl, decyl, etc. Alkyl groups can be unsubstituted or substituted. For example, “substituted alkyl” groups can be substituted with one or more moieties selected from halo, hydroxy, amino, alkylamino, alkoxy, haloalkyl, carboxy, amido, nitro, oxo, and cyano.

As used herein, the term “alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be unsubstituted or substituted. For example, “substituted alkenyl” groups can be substituted with one or more moieties selected from halo, hydroxy, amino, alkylamino, alkoxy, haloalkyl, carboxy, amido, nitro, oxo, and cyano.

As used herein, the term “alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be unsubstituted or substituted. For example, “substituted alkynyl” groups can be substituted with one or more moieties selected from halo, hydroxy, amino, alkylamino, alkoxy, haloalkyl, carboxy, amido, nitro, oxo, and cyano.

As used herein, the term “aryl” refers to an aromatic carbon ring system having any suitable number of ring atoms and any suitable number of rings. Aryl groups can include any suitable number of carbon ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be unsubstituted or substituted. For example, “substituted aryl” groups can be substituted with one or more moieties selected from halo, hydroxy, amino, alkylamino, alkoxy, haloalkyl, carboxy, amido, nitro, oxo, and cyano.

As used herein, the term “cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, and C₆₋₈. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbomene, and norbornadiene. Cycloalkyl groups can be unsubstituted or substituted. For example, “substituted cycloalkyl” groups can be substituted with one or more moieties selected from halo, hydroxy, amino, alkylamino, alkoxy, haloalkyl, carboxy, amido, nitro, oxo, and cyano.

As used herein, the term “heterocyclyl” refers to a saturated ring system having from 3 to 12 ring members and from 1 to 4 heteroatoms selected from N, O and S. Additional heteroatoms including, but not limited to, B, Al, Si and P can also be present in a heterocycloalkyl group. The heteroatoms can be oxidized to form moieties such as, but not limited to, —S(O)— and —S(O)₂—. Heterocyclyl groups can include any number of ring atoms, such as, 3 to 6, 4 to 6, 5 to 6, 4 to 6, or 4 to 7 ring members. Any suitable number of heteroatoms can be included in the heterocyclyl groups, such as 1, 2, 3, or 4, or 1 to 2, 1 to 3, 1 to 4, 2 to 3, 2 to 4, or 3 to 4. Examples of heterocyclyl groups include, but are not limited to, aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. Heterocyclyl groups can be unsubstituted or substituted. For example, “substituted heterocyclyl” groups can be substituted with one or more moieties selected from halo, hydroxy, amino, alkylamino, alkoxy, haloalkyl, carboxy, amido, nitro, oxo, and cyano.

As used herein, the term “heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms including, but not limited to, B, Al, Si and P can also be present in a heteroaryl group. The heteroatoms can be oxidized to form moieties such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. Examples of heteroaryl groups include, but are not limited to, pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. Heteroaryl groups can be unsubstituted or substituted. For example, “substituted heteroaryl” groups can be substituted with one or more moieties selected from halo, hydroxy, amino, alkylamino, alkoxy, haloalkyl, carboxy, amido, nitro, oxo, and cyano.

As used herein, the term “alkoxy” refers to an alkyl group having an oxygen atom that connects the alkyl group to the point of attachment: i.e., alkyl-O—. As for alkyl group, alkoxy groups can have any suitable number of carbon atoms, such as C₁₋₆ or C₁₋₄. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. Alkoxy groups can be unsubstituted or substituted. For example, “substituted alkoxy” groups can be substituted with one or more moieties selected from halo, hydroxy, amino, alkylamino, alkoxy, haloalkyl, carboxy, amido, nitro, oxo, and cyano.

As used herein, the term “alkylthio” refers to an alkyl group having a sulfur atom that connects the alkyl group to the point of attachment: i.e., alkyl-S—. As for alkyl groups, alkylthio groups can have any suitable number of carbon atoms, such as C₁₋₆ or C₁₋₄. Alkylthio groups include, for example, methoxy, ethoxy, propoxy, iso-propoxy, butoxy, 2-butoxy, iso-butoxy, sec-butoxy, tert-butoxy, pentoxy, hexoxy, etc. groups can be unsubstituted or substituted. For example, “substituted alkylthio” groups can be substituted with one or more moieties selected from halo, hydroxy, amino, alkylamino, alkoxy, haloalkyl, carboxy, amido, nitro, oxo, and cyano.

As used herein, the terms “halo” and “halogen” refer to fluorine, chlorine, bromine and iodine.

As used herein, the term “haloalkyl” refers to an alkyl moiety as defined above substituted with at least one halogen atom.

As used herein, the term “alkylsilyl” refers to a moiety —SiR₃, wherein at least one R group is alkyl and the other R groups are H or alkyl. The alkyl groups can be substituted with one or more halogen atoms.

As used herein, the term “acyl” refers to a moiety —C(O)R, wherein R is an alkyl group.

As used herein, the term “oxo” refers to an oxygen atom that is double-bonded to a compound (i.e., O═).

As used herein, the term “carboxy” refers to a moiety —C(O)OH. The carboxy moiety can be ionized to form the carboxylate anion. “Alkyl carboxylate” refers to a moiety —C(O)OR, wherein R is an alkyl group as defined herein.

As used herein, the term “amino” refers to a moiety —NR₃, wherein each R group is H or alkyl.

As used herein, the term “amido” refers to a moiety —NRC(O)R or —C(O)NR₂, wherein each R group is H or alkyl.

DNA methylation is an epigenetic modification carried out by methyltransferase enzymes that adds a methyl group to the 5-position of cytosine bases within genomic DNA, typically in CpG islands. This methyl group can be further modified to hydroxymethyl cytosine (addition of a single hydroxyl moiety), another epigenetic modification that is of growing scientific interest. These epigenetic markers provide additional, non-genetic regulation of genetic markers within the genome by suppressing or activating gene expression, depending on the genomic location of the methylation event. Due to their role in gene silencing or activation, dysregulation of methylation plays a crucial role in amplifying disease states, including cancer, diabetes, and other diseases that impact human health and wellbeing. Accordingly, assessing human health via sequencing is greatly improved by combining standard genome sequencing with novel sequencing strategies that identify the locations of these epigenetic markers

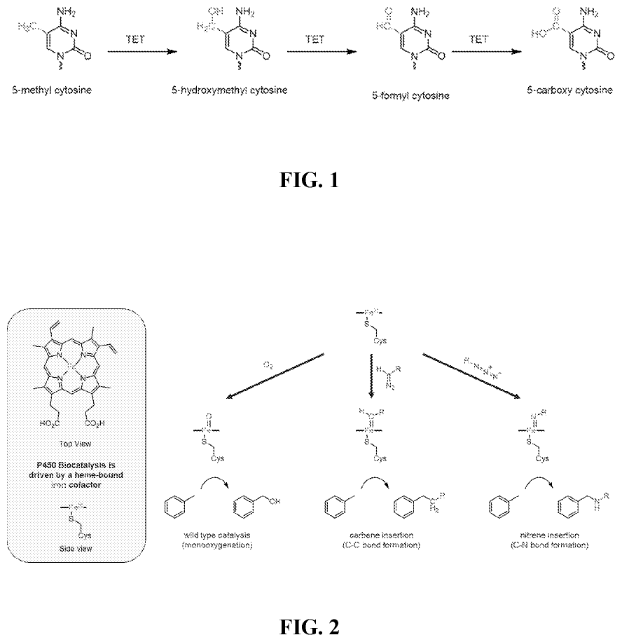

A number of chemical, enzymatic and chemoenzymatic strategies have been developed for the detection of DNA methylation events. The most common method currently used is bisulfite conversion which takes advantage of selective bisulfite-mediated deamination of cytosine to Uracil. Upon conversion and DNA replication, C is converted to T and this change can be observed via sequencing against a reference genome. Bisulfite is selective for cytosine and does not convert MeC or HO-MeC, thus these epigenetic markers appear as Cs during sequencing. However, bisulfite conversion is slow and destructive and can damage genomic DNA during library preparation. Since typically only 1-5% of the genome contains epigenetic MeC adducts, this method reduces the genome to a “3-base” genome, where most of the genome is T, G, or A (only a small fraction is C), which complicates data processing and necessitates the need for doping in large amounts of reference genomes like PhiX spike-ins to enable sequencing. Method EM-Seq provides an enzymatic (two enzyme) alternative to bisulfite sequencing, in which MeC is protected via oxidation to 5-carboxy cytosine using TET enzyme (FIG. 1). Then, a cytosine deaminase is added to enzymatically deaminate cytosine to uracil (similar to the role that bisulfite carries out above.) APOBEC has a broad substrate profile that permits deamination of C to U, but also MeC and HO-MeC to T and hydroxyT, respectively. However, APOBEC does not recognize 5-carboxy cytosine, thus TET-mediated oxidation protects these epigenetic markers enabling their detection via sequencing. EM-seq has various disadvantages, for example while the method is more mild than bisulfite sequencing, it remains a 3-base sequencing method. Also, TET oxidation is not homogeneous (FIG. 1) and can lead to a mixture of HO-MeC, 5-formylC and 5-carboxyC. Therefore, conditions must be optimized to push the reaction to completion. The Taps method is a four-base sequencing method. Similar to EM-Seq, methylation adducts are first converted to carboxy cytosine via TET oxidation in Tags, which is followed by chemical reduction by a borane reagent selectively reduces and decarboxylates 5-carboxy cytosine to dihydrouracil. However, Taps still has the need for complete conversion to 5-carboxy cytosine (intermediate oxidation states do not work), and has the issue of potential toxicity of the borane reductant.

Disclosed herein include a single enzyme method for the direct modification of methylcytosine and hydroxycytosine that is compatible with four base sequencing and provides a simplified solution for methylcytosine detection, as well as compositions, kits, and systems for performing the method. The method includes, in some embodiments, a one-step chemoenzymatic modification of MeC that leads to a direct readout of MeC adducts (as Ts) in sequencing (e.g., next generation sequencing). The method can, for example, significantly simplify methylomic library prep using an enzymatic reagent that is already in use by other MeC library prep kits.

### Reaction Mixtures for Performing Carbene-Insertion Reaction

Provided herein are reaction mixtures and methods for performing a TET-mediated carbene insertion in the 5-methyl moiety of the 5mC and/or the 5-hydroxymethyl moiety of 5hmC in a nucleic acid sequence.

The reaction mixture disclosed herein for performing a (TET)-mediated carbene insertion in 5-methylcytosine (5mC) 5-hydroxymethylcytosine (5hmC) comprise a nucleic acid suspected of comprising, or comprising, one or more 5-methylcytosine (5mC) or 5-hydroxymethylcytosine (5hmC), a carbene precursor for producing a C—H insertion in the 5-methyl moiety of the 5mC or the 5-hydroxymethyl moiety of 5hmC, and a TET or a variant thereof.

The term “carbene precursor” includes molecules that can be decomposed in the presence of metal (or enzyme) catalysts to form structures that contain at least one divalent carbon with two unshared valence shell electrons (i.e., carbenes) and that can be transferred to a carbon-hydrogen bond form of various carbon ligated products. Examples of carbene precursors include, but are not limited to, diazo reagents, diazirine reagents, and hydrazone reagents.

A number of carbene precursors can be used herein including, but not limited to, amines, azides, hydrazines, hydrazones, epoxides, diazirines, and diazo reagents. In some embodiments, the carbene precursor is an epoxide (i.e., a compound containing an epoxide moiety). The term “epoxide moiety” refers to a three-membered heterocycle having two carbon atoms and one oxygen atom connected by single bonds. In some embodiments, the carbene precursor is a diazirine (i.e., a compound containing a diazirine moiety). The term “diazirine moiety” refers to a three-membered heterocycle having one carbon atom and two nitrogen atoms, wherein the nitrogen atoms are connected via a double bond. Diazirines are chemically inert, small hydrophobic carbene precursors described, for example, in US 2009/0211893, by Turro (J. Am. Chem. Soc. 1987, 109, 2101-2107), and by Brunner (J Biol. Chem. 1980, 255, 3313-3318), which are incorporated herein by reference in their entirety.

In some embodiments, the carbene precursor is a diazo reagent, e.g., an α-diazoester, an α-diazoamide, an α-diazonitrile, an α-diazoketone, an α-diazoaldehyde, or an α-diazosilane. Diazo reagents can be formed from a number of starting materials using procedures that are known to those of skill in the art. Ketones (including 1,3-diketones), esters (including β-ketones), acyl chlorides, and carboxylic acids can be converted to diazo reagents employing diazo transfer conditions with a suitable transfer reagent (e.g., aromatic and aliphatic sulfonyl azides, such as toluenesulfonyl azide, 4-carboxyphenylsulfonyl azide, 2-naphthalenesulfonyl azide, methylsulfonyl azide, and the like) and a suitable base (e.g., triethylamine, triisopropylamine, diazobicyclo[2.2.2]octane, 1,8-diazabicyclo[5.4.0]undec-7-ene, and the like) as described, for example, in U.S. Pat. No. 5,191,069 and by Davies (J. Am. Chem. Soc. 1993, 115, 9468-9479), which are incorporated herein by reference in their entirety. The preparation of diazo compounds from azide and hydrazone precursors is described, for example, in U.S. Pat. Nos. 8,350,014 and 8,530,212, which are incorporated herein by reference in their entirety. Alkylnitrite reagents (e.g., β-methylbutyl)nitrite) can be used to convert α-aminoesters to the corresponding diazo compounds in non-aqueous media as described, for example, by Takamura (Tetrahedron, 1975, 31: 227), which is incorporated herein by reference in its entirety. Alternatively, a diazo compound can be formed from an aliphatic amine, an aniline or other arylamine, or a hydrazine using a nitrosating agent (e.g., sodium nitrite) and an acid (e.g., p-toluenesulfonic acid) as described, for example, by Zollinger (Diazo Chemistry I and II, VCH Weinheim, 1994) and in US 2005/0266579, which are incorporated herein by reference in their entirety.

In some embodiments, the carbene precursor has a structure of Formula I:

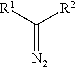

wherein
R¹ is selected from the group consisting of H, —C(O)OR^{1a}, —C(O)R^{1a}, C(O)N(R^{1b})₂, —SO₂R^{1a}, —SO₂OR¹, —P(O)(OR^{1a})₂, —NO₂, —CN, C₁₋₁₈ alkyl, C₂₋₁₈ alkenyl, C₂₋₁₈ alkynyl, 2- to 18-membered heteroalkyl, C₁₋₁₈ haloalkyl, C₁₋₁₈ alkoxy, C₃₋₁₀cycloalkyl, C₆₋₁₀ aryl, 3- to 10-membered heterocyclyl, and 5- to 10-membered heteroaryl;
each R^{1a} is independently selected from the group consisting of H, C₁₋₁₈ alkyl, C₂₋₁₈ alkenyl, C₂₋₁₈ alkynyl, C₆₋₁₀ aryl, 3- to 10-membered heterocyclyl, and 5- to 10-membered heteroaryl;
each R^{1b} is independently selected from the group consisting of H, C₁₋₁₈ alkyl, C₂₋₁₈ alkenyl, C₂₋₁₈ alkynyl, and C₁₋₁₈ alkoxy;
R² is an electron-withdrawing group selected from the group consisting of —C(O)OR^{2a}, —C(O)R^{2a}, —C(O)N(R^{2b})₂, —SO₂R^{2a}, —SO₂OR^{2a}, —P(O)(OR^{2a})₂, —NO₂, and —CN;
each R^{2a} is independently selected from the group consisting of H, C₁₋₁₈ alkyl, C₂₋₁₈ alkenyl, C₂₋₁₈ alkynyl, C₆₋₁₀ aryl, 3- to 10-membered heterocyclyl, and 5- to 10-membered heteroaryl;
each R^{2b} is independently selected from the group consisting of H, C₁₋₁₈ alkyl, C₂₋₁₈ alkenyl, C₂₋₁₈ alkynyl, and C₁₋₈ alkoxy; and
R¹ and R² are optionally and independently substituted; or
R¹ and R² are taken together to form C₃₋₁₀ cycloalkyl, C₆₋₁₀ aryl, 3- to 10-membered heterocyclyl, and 5- to 10-membered heteroaryl, each of which is optionally substituted.

In some embodiments, the carbene precursor is a compound according to Formula I wherein:
R¹ is selected from the group consisting of H, —C(O)OR^{1a}, —C(O)R^{1a}, —C(O)N(R^{1b})₂, —SO₂Ria, —SO₂OR^{1a}, —P(O)(OR^{1a})₂, —NO₂, —CN, C₁₋₁₈ alkyl, 2- to 18-membered heteroalkyl, C₁₋₁₈haloalkyl, C₁₋₁₈alkoxy, C₃₋₁₀cycloalkyl, C₆₋₁₀aryl, 3- to 10-membered heterocyclyl, and 5- to 10-membered heteroaryl;
each R^{1a} is independently C₁₋₈ alkyl;
each R^{1b} is independently selected from the group consisting of H, C₁₋₈ alkyl, and C₁₋₈ alkoxy;
R² is an electron-withdrawing group selected from the group consisting of —C(O)OR^{2a}, —C(O)R^{2a}, —C(O)N(R^{2b})₂, —SO₂R^{2a}, —SO₂OR^{2a}, —P(O)(OR^{2a})₂, —NO₂, and —CN;
each R^{2a} is independently C₁₋₈ alkyl;
each R^{2b} is independently selected from the group consisting of H, C₁₋₈ alkyl, and C₁₋₈ alkoxy; and
R¹ and R² are optionally and independently substituted; or
R¹ and R² are taken together to form C₃₋₁₀ cycloalkyl, C₆₋₁₀ aryl, 3- to 10-membered heterocyclyl, and 5- to 10-membered heteroaryl, each of which is optionally substituted.

In some embodiments, the carbene precursor is a compound according to Formula I wherein
R¹ is independently selected from the group consisting of H, —C(O)OR^{1a}, C(O)R^{1a}, —SO₂R^{1a}, —SO₂OR^{1a}, substituted C₁₋₁₈ alkyl, 2- to 18-membered heteroalkyl, C₁₋₁₈ alkoxy, C₃₋₁₀ cycloalkyl, C₁₋₁₈ fluoroalkyl, substituted C₆₋₁₀ aryl, and substituted 5- to 10-membered heteroaryl;
R^{1a} is C₁₋₈ alkyl;
R² is selected from the group consisting of —C(O)OR^{2a}, —C(O)R^{2a}, —SO₂R^{2a}, and —SO₂OR^{2a}; and
R^{2a} is C₁₋₈ alkyl; or
R¹ and R² are optionally taken together to form C₃₋₁₀ cycloalkyl, C₆₋₁₀ aryl, 3- to 10-membered heterocyclyl, and 5- to 10-membered heteroaryl, each of which is optionally substituted.

In some embodiments, R² is —C(O)OR^{2a} or —C(O)N(R^{2b})₂. In some embodiments, R² is —C(O)OR^{2a} and R^{2a} is C₁₋₈ alkyl or C₁₋₈ alkyl substituted with C₆₋₁₀aryl. R^{2a} can be further substituted with one or more substituents (e.g., 1-6 substituents, or 1-3 substituents, or 1-2 substituents) independently selected from halogen, —OH, —NO₂; —CN; —N3; C₁₋₆ alkyl, C₁₋₆ alkoxy, C₁₋₆ haloalkyl, C₁₋₁₈ alkylsilyl, unsubstituted C₆₋₁₀ aryl, and substituted C₆₋₁₀aryl. In some embodiments, R² is —C(O)OR^{2a} and R¹ is H, C₁₋₈ alkyl, C₁₋₁₈ alkoxy, C₃₋₁₀ cycloalkyl, or C₆₋₁₀ aryl. In some such embodiments, R¹ is H or C₁₋₈ alkyl.

In some embodiments, R² is —C(O)N(R^{2b})₂ and each R^{2b} is independently C₁₋₈ alkyl or C₁₋₈ alkoxy. In some such embodiments, R¹ is H, C₁₋₈ alkyl, C₁₋₁₈ alkoxy, C₃₋₁₀ cycloalkyl, or C₆₋₁₀ aryl. In some embodiments, R¹ is H or C₁₋₈ alkyl.

In some embodiments, R² and R¹ are taken together with the central carbon atom in Formula I to form C₃₋₁₀ cycloalkyl, C₆₋₁₀ aryl, 3- to 10-membered heterocyclyl, or 5- to 10-membered heteroaryl. In some embodiments, R² is C(O)OR^{2a}, —C(O)R^{2a}, or —C(O)N(R^{2b})₂, wherein R^{2a} or one R^{2b} is taken together with R¹ to form C₃₋₁₀ cycloalkyl or 3- to 10-membered heterocyclyl. For example, R^{2a} and R¹ can be taken together to form dihydrofuran-2(3H)-one when the carbene precursor according to Formula I is 3-diazodihydrofuran-2(3H)-one.

In some embodiments, the carbene precursor is selected from the group consisting of diazo reagents, diazirine reagents, hydrazone reagents, and a combination thereof. In some embodiments, the carbene precursor is selected from the group consisting of:

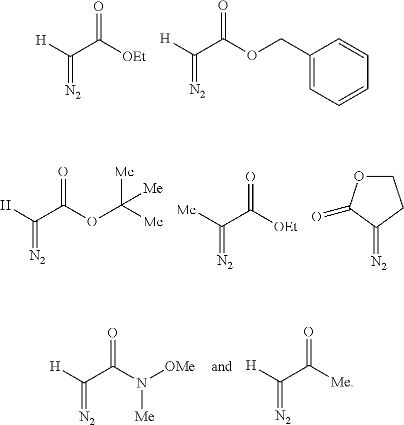

wherein “Me” denotes a methyl group and “Et” denotes an ethyl group.

In some embodiments, the carbene precursor is diazoacetate ester.

Reaction mixtures disclosed herein can contain additional reagents. The additional reagents include, but not limited to, buffers (e.g., M9-N buffer, 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, isopropanol, glycerol, tetrahydrofuran, acetone, acetonitrile, and acetic acid), salts (e.g., NaCl, KCl, CaCl₂), and salts of Mn²⁺ and Mg²⁺), denaturants (e.g., urea and guanadinium hydrochloride), detergents (e.g., sodium dodecylsulfate and Triton-X 100), chelators (e.g., ethylene glycol-bis(2-aminoethylether)-N,N,N′,N′-tetraacetic acid (EGTA), 2-({2-[Bis(carboxymethyl)amino]ethyl} (carboxymethyl)amino)acetic acid (EDTA), and 1,2-bis(o-aminophenoxy)ethane-N,N,N′,N′-tetraacetic acid (BAPTA)), sugars (e.g., glucose, sucrose, and the like), and reducing agents (e.g., sodium dithionite, NADPH, dithiothreitol (DTT), β-mercaptoethanol (BME), and tris(2-carboxyethyl)phosphine (TCEP)). Buffers, cosolvents, salts, denaturants, detergents, chelators, sugars, and reducing agents can be used at any suitable concentration, which can be readily determined by one of skill in the art.

In the methods and compositions disclosed herein, buffers, cosolvents, salts, denaturants, detergents, chelators, sugars, and reducing agents, if present, are included in reaction mixtures at concentrations ranging from about 1 μM to about 1 M (including 1 μM, 5 μM, 10 μM, 20 μM, 50 μM, 100 μM, 200 μM, 500 μM, 1 mM, 10 mM, 50 mM, 100 mM, 500 mM, 1M, a number within any of these values, or a range between any two of these values). For example, a buffer, a cosolvent, a salt, a denaturant, a detergent, a chelator, a sugar, or a reducing agent can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In some embodiments, a reducing agent is used in a sub-stoichiometric amount. Cosolvents, in particular, can be included in the reaction mixtures in amounts ranging from about 1% v/v to about 75% v/v, or higher. A cosolvent can be included in the reaction mixture, for example, in an amount of about 5, 10, 20, 30, 40, or 50% (v/v).

Reactions are conducted under conditions sufficient to catalyze a carbene insertion in a nucleic acid comprising 5-methylcytosine (5mC), 5-hydroxymethylcytosine (5hmC) or both. For example, the reactions can be conducted at any suitable temperature. In general, the reactions are conducted at a temperature of from about 0° C. to about 40° C. The reactions can be conducted, for example, at about 25° C. or about 37° C. In certain embodiments, high stereoselectivity can be achieved by conducting the reaction at a temperature less than 25° C. (e.g., about 20° C., 10° C., or 4° C.) without reducing the total turnover number of the enzyme catalyst. The reactions can be conducted at any suitable pH. In general, the reactions are conducted at a pH of from about 6 to about 10. The reactions can be conducted, for example, at a pH of from about 6.5 to about 9 (e.g., about pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, or a range between any two of these values). The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 18 hours, or about 24 hours, or about 48 hours, or about 72 hours. In some embodiments, the reaction is conducted for a period of time ranging from about 6 hours to about 24 hours (e.g., about 6, 7, 8, 9, 10, 11, 12, 13, 14, 15, 16, 17, 18, 19, 20 21, 22, 23, 24 hours, or a range between any two of these values).

The reaction mixtures disclosed herein can be used for reactions conducted under aerobic conditions or anaerobic conditions.

The TET-mediated carbene insertion reaction disclosed herein on the 5-methyl moiety of the 5mC or the 5-hydroxymethyl moiety of 5hmC in a target nucleic acid to generate a modified target nucleic acid can occur in vitro, in vivo or ex vivo. For example, a TET enzyme (e.g., a recombinant TET) can be expressed in a host cell, thereby the 5-methyl moiety of the 5mC or the 5-hydroxymethyl moiety of 5hmC in nucleic acids in the host cell can be modified by the TET enzyme (e.g., the recombinant TET) to generate modified nucleic acids, for example converting the 5mC or 5hmC into a modified nucleic acid adduct capable of forming a hydrogen bond with adenine (A). In some embodiments, a TET enzyme (e.g., a recombinant TET enzyme) is introduced into a host cell, thereby the 5-methyl moiety of the 5mC or the 5-hydroxymethyl moiety of 5hmC in nucleic acids in the host cell can be modified by the TET enzyme to generate modified nucleic acids, for example converting the 5mC or 5hmC into a modified nucleic acid adduct capable of forming a hydrogen bond with adenine (A).

The reaction mixtures disclosed herein can be used for a reaction under anaerobic conditions, thereby diverting the natural TET-mediate oxidation of MeC to HO-MeC into a non-natural carbene-insertion reaction in the 5-methyl moiety of 5-mC or the 5-hydroxymethyl moiety of 5-hmC by removing oxygen. The term “anaerobic” when used in reference to a reaction, culture or growth condition, is intended to mean that the concentration of oxygen is less than about 25 μM, preferably less than about 5 μM, and even more preferably less than 1 μM. The term is also intended to include sealed chambers of liquid or solid medium maintained with an atmosphere of less than about 1% oxygen. Reactions can be conducted under an inert atmosphere, such as a nitrogen atmosphere or argon atmosphere, by sparging a reaction mixture with an inert gas such as nitrogen or argon.

The reaction mixtures disclosed herein can also be used for a reaction under aerobic conditions. The term “aerobic” when used in reference to a reaction, culture or growth condition, is intended to mean that the concentration of oxygen is greater than about 25 μM, preferably greater than about 100 μM, and even more preferably less than 1 mM. The reaction mixtures can further comprise a non-reducing acid or a salt thereof to divert the natural TET-mediate oxidation of MeC to HO-MeC into a non-natural carbene-insertion reaction in the 5-methyl moiety of 5-mC or the 5-hydroxymethyl moiety of 5-hmC. The term “non-reducing acid” refers to acids having low ability to oxidize or reduce other substances, in other words reluctant to accept or donate electrons. Non-reducing acid include organic acids such as acetic acid, dichloroacetic acid, fluoroacetic acid, chloroacetic acid, citric acid, ascorbic acid, benzoic acid, N-oxalylglycine, succinic acid, 2-pyridine carboxylic acid, 2,4-pyridine dicarboxylic acid (2,4-PDCA), 5-carboxy-8-hydroxyquinoline, FG-2216, FG-4592, and a combination thereof.

The concentration of the nucleic acid comprising one or more 5-methylcytosine (5mC) or 5-hydroxymethylcytosine (5hmC), a carbene precursor, and/or a non-reducing acid or a salt thereof in the reaction mixture can vary, for example from about 100 μM to about 1 M. The concentration can be, for example, from about 100 μM to about 1 mM, or about from 1 mM to about 100 mM, or from about 100 mM to about 500 mM, or from about 500 mM to 1 M. The concentration can be from about 500 μM to about 500 mM, 500 μM to about 50 mM, or from about 1 mM to about 50 mM, or from about 15 mM to about 45 mM, or from about 15 mM to about 30 mM, or from about 5 mM to about 25 mM, or from about 5 mM to about 15 mM.

In embodiments herein described, the reaction mixtures disclosed herein carry out a non-natural TET-medicated reaction that is diverted from its natural oxidation reaction. The non-natural reaction results in a carbene-insertion in the 5-methyl moiety of 5mC or the 5-hydroxymethyl moiety of 5hmC, thereby generating a modified nucleic acid base that can form a hydrogen bond with adenine (A) and thus read directly as or copied to Thymine (T) via polymerase chain reaction.

### TET Enzymes and Variants

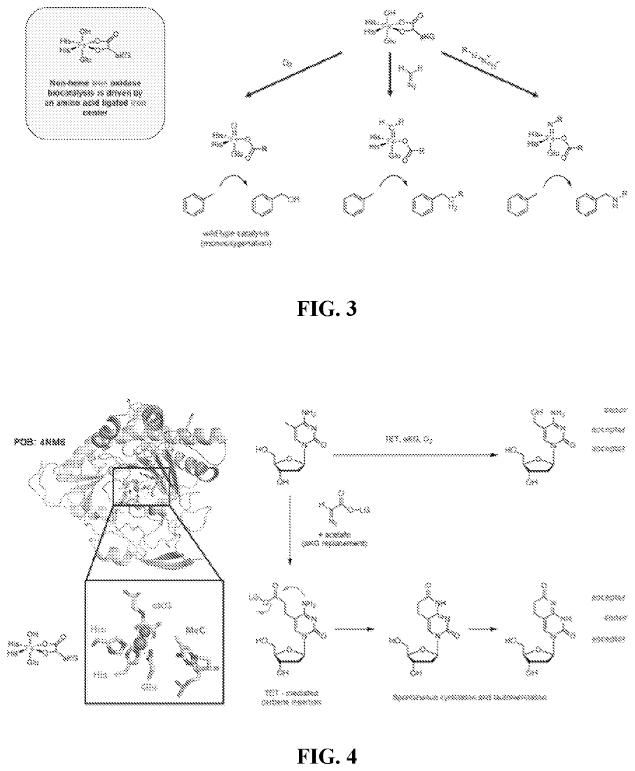

Disclosed herein include TET proteins and a variants thereof “TET” or “ten eleven translocation enzyme” used herein refers to a family of enzymes of ten-eleven translocation (TET) methylcytosine dioxygenases. The TET enzyme can, for example catalyze, in a natural reaction condition, the iterative demethylation of 5mC. The transfer of an oxygen molecule to the N5 methyl group on 5mC resulting in the formation of 5-hydroxymethylcytosine (5hmC). TET further catalyzes the oxidation of 5hmC to 5-formylC (5fC) and the oxidation of 5fC to form 5-carboxyC (5caC). TET is a non-heme iron oxygenase that can carry out oxidation of MeC using an enzyme bound iron catalyst, a small molecule cofactor (alpha-ketoglutarate, aKG) for iron reduction, and molecular oxygen as the oxygenation source. The key feature of this family of enzymes is the iron center, which is the active catalyst for these enzymes. Similar chemistry is observed in other enzymes, including heme-containing proteins such as globins and cytochrome P450s (FIGS. 2 and 3).

The TET enzymes described herein contain a conserved double-stranded (3-helix (DSBH) domain, a cysteine-rich domain, and binding sites for cofactors Fe(II) and α-ketoglutaric acid that together form the core catalytic region in the C-terminus. In some embodiments of the TET or variants used herein, the natural reducing cofactor α-ketoglutaric acid is absent. The α-ketoglutaric acid in the TET enzymes used herein can be replaced by a non-reducing acid described above. The non-reducing acid can be one or more organic acids such as acetic acid, dichloroacetic acid, fluoroacetic acid, chloroacetic acid, citric acid, ascorbic acid, benzoic acid, and a combination thereof.

The TET enzyme used herein can be, for example, one or more of human TET1, TET2, TET3, and variants thereof, murine Tet1, Tet2, Tet3, and variants thereof, Naegleria TET (NgTET, e.g., Naegleria gruberi TET) and variants thereof, Coprinopsis cinerea (CcTET) and variants thereof, and a combination thereof. In some embodiments, the TET enzyme is human TET1. In some embodiments, the TET enzyme is NgTET. The TET enzyme can be, for example, a prokaryotic TET enzyme or a eukaryotic TET enzyme. In some embodiments, the TET enzyme is a viral TET enzyme, for example a bacteriophage TET. Non-limiting examples of phase-encoded TET are described in, for example, Burket et al. PNAS Jun. 29, 2021 118 (26) e2026742118, the content of which is hereby expressly incorporated by references.

Exemplary TET proteins include, for example, human TET1 of SEQ ID: 1, human TET2 of SEQ ID NO: 2, human TET3 of SEQ ID NO: 3, murine Tet1 of SEQ ID NO: 4, murine Tet2 of SEQ ID NO: 5, murine Tet3 of SEQ ID NO: 6, NgTET of SEQ ID NO: 7, and other TET proteins deposited in public databases such as GeneBank or UniProt identifiable to a person skilled in the art. Table 1 provides a non-limiting list of exemplary TET protein sequences. Table 1: A non-limiting list of exemplary TET protein sequences.

TABLE 1
A non-limiting list of exemplary TET protein sequences

| Name | Sequence | SEQ ID NO |
|---|---|---|
| Human | MSRSRHARPSRLVRKEDVNKKKKNSQLRKTTKGANKNVASVKT | 1 |
| TET1 | LSPGKLKQLIQERDVKKKTEPKPPVPVRSLLTRAGAARMNLDR | |
| | TEVLFQNPESLTCNGFTMALRSTSLSRRLSQPPLVVAKSKKVP | |
| | LSKGLEKQHDCDYKILPALGVKHSENDSVPMQDTQVLPDIETL | |
| | IGVQNPSLLKGKSQETTQFWSQRVEDSKINIPTHSGPAAEILP | |
| | GPLEGTRCGEGLFSEETLNDTSGSPKMFAQDTVCAPFPQRATP | |
| | KVTSQGNPSIQLEELGSRVESLKLSDSYLDPIKSEHDCYPTSS | |
| | LNKVIPDLNLRNCLALGGSTSPTSVIKELLAGSKQATLGAKPD | |
| | HQEAFEATANQQEVSDTTSFLGQAFGAIPHQWELPGADPVHGE | |
| | ALGETPDLPEIPGAIPVQGEVFGTILDQQETLGMSGSVVPDLP | |
| | VFLPVPPNPIATFNAPSKWPEPQSTVSYGLAVQGAIQILPLGS | |
| | GHTPQSSSNSEKNSLPPVMAISNVENEKQVHISFLPANTQGFP | |
| | LAPERGLFHASLGIAQLSQAGPSKSDRGSSQVSVTSTVHVVNT | |
| | TVVTMPVPMVSTSSSSYTTLLPTLEKKKRKRCGVCEPCQQKTN | |
| | CGECTYCKNRKNSHQICKKRKCEELKKKPSVVVPLEVIKENKR | |
| | PQREKKPKVLKADFDNKPVNGPKSESMDYSRCGHGEEQKLELN | |
| | PHTVENVTKNEDSMTGIEVEKWTQNKKSQLTDHVKGDESANVP | |
| | EAEKSKNSEVDKKRTKSPKLFVQTVRNGIKHVHCLPAETNVSF | |
| | KKFNIEEFGKTLENNSYKFLKDTANHKNAMSSVATDMSCDHLK | |
| | GRSNVLVFQQPGENCSSIPHSSHSIINHHASIHNEGDQPKTPE | |
| | NIPSKEPKDGSPVQPSLLSLMKDRRLTLEQVVAIEALTQLSEA | |
| | PSENSSPSKSEKDEESEQRTASLLNSCKAILYTVRKDLQDPNL | |
| | QGEPPKLNHCPSLEKQSSCNTVVENGQTTTLSNSHINSATNQA | |
| | STKSHEYSKVINSLSLFIPKSNSSKIDTNKSIAQGIITLDNCS | |
| | NDLHQLPPRNNEVEYCNQLLDSSKKLDSDDLSCQDATHTQIEE | |
| | DVATQLTQLASIIKINYIKPEDKKVESTPTSLVTCNVQQKYNQ | |
| | EKGTIQQKPPSSVHNNHGSSLTKQKNPTQKKTKSTPSRDRRKK | |
| | KPTVVSYQENDRQKWEKLSYMYGTICDIWIASKFQNFGQFCPH | |
| | DFPTVFGKISSSTKIWKPLAQTRSIMQPKTVFPPLTQIKLQRY | |
| | PESAEEKVKVEPLDSLSLFHLKTESNGKAFTDKAYNSQVQLTV | |
| | NANQKAHPLTQPSSPPNQCANVMAGDDQIRFQQVVKEQLMHQR | |
| | LPTLPGISHETPLPESALTLRNVNVVCSGGITVVSTKSEEEVC | |
| | SSSFGTSEFSTVDSAQKNFNDYAMNFFTNPTKNLVSITKDSEL | |
| | PTCSCLDRVIQKDKGPYYTHLGAGPSVAAVREIMENRYGQKGN | |
| | AIRIEIVVYTGKEGKSSHGCPIAKWVLRRSSDEEKVLCLVRQR | |
| | TGHHCPTAVMVVLIMVWDGIPLPMADRLYTELTENLKSYNGHP | |
| | TDRRCTLNENRTCTCQGIDPETCGASFSFGCSWSMYENGCKFG | |
| | RSPSPRRFRIDPSSPLHEKNLEDNLQSLATRLAPIYKQYAPVA | |
| | YQNQVEYENVARECRLGSKEGRPFSGVTACLDFCAHPHRDIHN | |
| | MNNGSTVVCTLTREDNRSLGVIPQDEQLHVLPLYKLSDTDEFG | |
| | SKEGMEAKIKSGAIEVLAPRRKKRTCFTQPVPRSGKKRAAMMT | |
| | EVLAHKIRAVEKKPIPRIKRKNNSTTTNNSKPSSLPTLGSNTE | |
| | TVQPEVKSETEPHFILKSSDNTKTYSLMPSAPHPVKEASPGES | |
| | WSPKTASATPAPLKNDATASCGFSERSSTPHCTMPSGRLSGAN | |
| | AAAADGPGISQLGEVAPLPTLSAPVMEPLINSEPSTGVTEPLT | |
| | PHQPNHQPSFLTSPQDLASSPMEEDEQHSEADEPPSDEPLSDD | |
| | PLSPAEEKLPHIDEYWSDSEHIFLDANIGGVAIAPAHGSVLIE | |
| | CARRELHATTPVEHPNRNHPTRLSLVFYQHKNLNKPQHGFELN | |
| | KIKFEAKEAKNKKMKASEQKDQAANEGPEQSSEVNELNQIPSH | |
| | KALTLTHDNVVTVSPYALTHVAGPYNHWV | |
| Human | MEQDRTNHVEGNRLSPFLIPSPPICQTEPLATKLQNGSPLPER | 2 |
| TET2 | AHPEVNGDTKWHSFKSYYGIPCMKGSQNSRVSPDFTQESRGYS | |
| | KCLQNGGIKRTVSEPSLSGLLQIKKLKQDQKANGERRNFGVSQ | |
| | ERNPGESSQPNVSDLSDKKESVSSVAQENAVKDFTSESTHNCS | |
| | GPENPELQILNEQEGKSANYHDKNIVLLKNKAVLMPNGATVSA | |
| | SSVEHTHGELLEKTLSQYYPDCVSIAVQKTTSHINAINSQATN | |
| | ELSCEITHPSHTSGQINSAQTSNSELPPKPAAVVSEACDADDA | |
| | DNASKLAAMLNTCSFQKPEQLQQQKSVFEICPSPAENNIQGTT | |
| | KLASGEEFCSGSSSNLQAPGGSSERYLKQNEMNGAYFKQSSVF | |
| | TKDSFSATTTPPPPSQLLLSPPPPLPQVPQLPSEGKSTLNGGV | |
| | LEEHHHYPNQSNTTLLREVKIEGKPEAPPSQSPNPSTHVCSPS | |
| | PMLSERPQNNCVNRNDIQTAGTMTVPLCSEKTRPMSEHLKHNP | |
| | PIFGSSGELQDNCQQLMRNKEQEILKGRDKEQTRDLVPPTQHY | |
| | LKPGWIELKAPRFHQAESHLKRNEASLPSILQYQPNLSNQMTS | |
| | KQYTGNSNMPGGLPRQAYTQKTTQLEHKSQMYQVEMNQGQSQG | |
| | TVDQHLQFQKPSHQVHFSKTDHLPKAHVQSLCGTRFHFQQRAD | |
| | SQTEKLMSPVLKQHLNQQASETEPFSNSHLLQHKPHKQAAQTQ | |
| | PSQSSHLPQNQQQQQKLQIKNKEEILQTFPHPQSNNDQQREGS | |
| | FFGQTKVEECFHGENQYSKSSEFETHNVQMGLEEVQNINRRNS | |
| | PYSQTMKSSACKIQVSCSNNTHLVSENKEQTTHPELFAGNKTQ | |
| | NLHHMQYFPNNVIPKQDLLHRCFQEQEQKSQQASVLQGYKNRN | |
| | QDMSGQQAAQLAQQRYLIHNHANVFPVPDQGGSHTQTPPQKDT | |
| | QKHAALRWHLLQKQEQQQTQQPQTESCHSQMHRPIKVEPGCKP | |
| | HACMHTAPPENKTWKKVTKQENPPASCDNVQQKSIIETMEQHL | |
| | KQFHAKSLFDHKALTLKSQKQVKVEMSGPVTVLTRQTTAAELD | |
| | SHTPALEQQTTSSEKTPTKRTAASVLNNFIESPSKLLDTPIKN | |
| | LLDTPVKTQYDFPSCRCVEQIIEKDEGPFYTHLGAGPNVAAIR | |
| | EIMEERFGQKGKAIRIERVIYTGKEGKSSQGCPIAKWVVRRSS | |
| | SEEKLLCLVRERAGHTCEAAVIVILILVWEGIPLSLADKLYSE | |
| | LTETLRKYGTLTNRRCALNEERTCACQGLDPETCGASESFGCS | |
| | WSMYYNGCKFARSKIPRKFKLLGDDPKEEEKLESHLQNLSTLM | |
| | APTYKKLAPDAYNNQIEYEHRAPECRLGLKEGRPESGVTACLD | |
| | FCAHAHRDLHNMQNGSTLVCTLTREDNREFGGKPEDEQLHVLP | |
| | LYKVSDVDEFGSVEAQEEKKRSGAIQVLSSERRKVRMLAEPVK | |
| | TCRQRKLEAKKAAAEKLSSLENSSNKNEKEKSAPSRTKQTENA | |
| | SQAKQLAELLRLSGPVMQQSQQPQPLQKQPPQPQQQQRPQQQQ | |
| | PHHPQTESVNSYSASGSTNPYMRRPNPVSPYPNSSHTSDIYGS | |
| | TSPMNFYSTSSQAAGSYLNSSNPMNPYPGLLNQNTQYPSYQCN | |
| | GNLSVDNCSPYLGSYSPQSQPMDLYRYPSQDPLSKLSLPPIHT | |
| | LYQPRFGNSQSFTSKYLGYGNQNMQGDGESSCTIRPNVHHVGK | |
| | LPPYPTHEMDGHFMGATSRLPPNLSNPNMDYKNGEHHSPSHII | |
| | HNYSAAPGMFNSSLHALHLQNKENDMLSHTANGLSKMLPALNH | |
| | DRTACVQGGLHKLSDANGQEKQPLALVQGVASGAEDNDEVWSD | |
| | SEQSFLDPDIGGVAVAPTHGSILIECAKRELHATTPLKNPNRN | |
| | HPTRISLVFYQHKSMNEPKHGLALWEAKMAEKAREKEEECEKY | |
| | GPDYVPQKSHGKKVKREPAEPHETSEPTYLRFIKSLAERTMSV | |
| | TTDSTVTTSPYAFTRVTGPYNRYI | |
| Human | MSQFQVPLAVQPDLPGLYDFPQRQVMVGSFPGSGLSMAGSESQ | 3 |
| TET3 | LRGGGDGRKKRKRCGTCEPCRRLENCGACTSCTNRRTHQICKL | |
| | RKCEVLKKKVGLLKEVEIKAGEGAGPWGQGAAVKTGSELSPVD | |
| | GPVPGQMDSGPVYHGDSRQLSASGVPVNGAREPAGPSLLGTGG | |
| | PWRVDQKPDWEAAPGPAHTARLEDAHDLVAFSAVAEAVSSYGA | |
| | LSTRLYETFNREMSREAGNNSRGPRPGPEGCSAGSEDLDTLQT | |
| | ALALARHGMKPPNCNCDGPECPDYLEWLEGKIKSVVMEGGEER | |
| | PRLPGPLPPGEAGLPAPSTRPLLSSEVPQISPQEGLPLSQSAL | |
| | SIAKEKNISLQTAIAIEALTQLSSALPQPSHSTPQASCPLPEA | |
| | LSPPAPERSPQSYLRAPSWPVVPPEEHSSFAPDSSAFPPATPR | |
| | TEFPEAWGTDTPPATPRSSWPMPRPSPDPMAELEQLLGSASDY | |
| | IQSVFKRPEALPTKPKVKVEAPSSSPAPAPSPVLQREAPTPSS | |
| | EPDTHQKAQTALQQHLHHKRSLFLEQVHDTSFPAPSEPSAPGW | |
| | WPPPSSPVPRLPDRPPKEKKKKLPTPAGGPVGTEKAAPGIKPS | |
| | VRKPIQIKKSRPREAQPLFPPVRQIVLEGLRSPASQEVQAHPP | |
| | APLPASQGSAVPLPPEPSLALFAPSPSRDSLLPPTQEMRSPSP | |
| | MTALQPGSTGPLPPADDKLEELIRQFEAEFGDSFGLPGPPSVP | |
| | IQDPENQQTCLPAPESPFATRSPKQIKIESSGAVTVLSTTCFH | |
| | SEEGGQEATPTKAENPLTPTLSGFLESPLKYLDTPTKSLLDTP | |
| | AKRAQAEFPTCDCVEQIVEKDEGPYYTHLGSGPTVASIRELME | |
| | ERYGEKGKAIRIEKVIYTGKEGKSSRGCPIAKWVIRRHTLEEK | |
| | LLCLVRHRAGHHCQNAVIVILILAWEGIPRSLGDTLYQELTDT | |
| | LRKYGNPTSRRCGLNDDRTCACQGKDPNTCGASESFGCSWSMY | |
| | FNGCKYARSKTPRKFRLAGDNPKEEEVLRKSFQDLATEVAPLY | |
| | KRLAPQAYQNQVTNEEIAIDCRLGLKEGRPFAGVTACMDFCAH | |
| | AHKDQHNLYNGCTVVCTLTKEDNRCVGKIPEDEQLHVLPLYKM | |
| | ANTDEFGSEENQNAKVGSGAIQVLTAFPREVRRLPEPAKSCRQ | |
| | RQLEARKAAAEKKKIQKEKLSTPEKIKQEALELAGITSDPGLS | |
| | LKGGLSQQGLKPSLKVEPQNHESSFKYSGNAVVESYSVLGNCR | |
| | PSDPYSMNSVYSYHSYYAQPSLTSVNGFHSKYALPSFSYYGEP | |
| | SSNPVFPSQFLGPGAWGHSGSSGSFEKKPDLHALHNSLSPAYG | |
| | GAEFAELPSQAVPTDAHHPTPHHQQPAYPGPKEYLLPKAPLLH | |
| | SVSRDPSPFAQSSNCYNRSIKQEPVDPLTQAEPVPRDAGKMGK | |
| | TPLSEVSQNGGPSHLWGQYSGGPSMSPKRTNGVGGSWGVFSSG | |
| | ESPAIVPDKLSSFGASCLAPSHFTDGQWGLFPGEGQQAASHSG | |
| | GRLRGKPWSPCKFGNSTSALAGPSLTEKPWALGAGDENSALKG | |
| | SPGFQDKLWNPMKGEEGRIPAAGASQLDRAWQSFGLPLGSSEK | |
| | LEGALKSEEKLWDPESLEEGPAEEPPSKGAVKEEKGGGGAEEE | |
| | EEELWSDSEHNFLDENIGGVAVAPAHGSILIECARRELHATTP | |
| | LKKPNRCHPTRISLVFYQHKNLNQPNHGLALWEAKMKQLAERA | |
| | RARQEEAARLGLGQQEAKLYGKKRKWGGTVVAEPQQKEKKGVV | |
| | PTRQALAVPTDSAVTVSSYAYTKVTGPYSRWI | |
| Murine | MSRSRPAKPSKSVKTKLQKKKDIQMKTKTSKQAVRHGASAKAV | 4 |
| Tet1 | NPGKPKQLIKRRDGKKETEDKTPTPAPSFLTRAGAARMNRDRN | |
| | QVLFQNPDSLTCNGFTMALRRTSLSWRLSQRPVVTPKPKKVPP | |
| | SKKQCTHNIQDEPGVKHSENDSVPSQHATVSPGTENGEQNRCL | |
| | VEGESQEITQSCPVFEERIEDTQSCISASGNLEAEISWPLEGT | |
| | HCEELLSHQTSDNECTSPQECAPLPQRSTSEVTSQKNTSNQLA | |
| | DLSSQVESIKLSDPSPNPTGSDHNGFPDSSFRIVPELDLKTCM | |
| | PLDESVYPTALIRFILAGSQPDVEDTKPQEKTLITTPEQVGSH | |
| | PNQVLDATSVLGQAFSTLPLQWGFSGANLVQVEALGKGSDSPE | |
| | DLGAITMLNQQETVAMDMDRNATPDLPIFLPKPPNTVATYSSP | |
| | LLGPEPHSSTSCGLEVQGATPILTLDSGHTPQLPPNPESSSVP | |
| | LVIAANGTRAEKQFGTSLFPAVPQGFTVAAENEVQHAPLDLTQ | |
| | GSQAAPSKLEGEISRVSITGSADVKATAMSMPVTQASTSSPPC | |
| | NSTPPMVERRKRKACGVCEPCQQKANCGECTYCKNRKNSHQIC | |
| | KKRKCEVLKKKPEATSQAQVTKENKRPQREKKPKVLKTDENNK | |
| | PVNGPKSESMDCSRRGHGEEEQRLDLITHPLENVRKNAGGMTG | |
| | IEVEKWAPNKKSHLAEGQVKGSCDANLTGVENPQPSEDDKQQT | |
| | NPSPTFAQTIRNGMKNVHCLPTDTHLPLNKLNHEEFSKALGNN | |
| | SSKLLTDPSNCKDAMSVTTSGGECDHLKGPRNTLLFQKPGLNC | |
| | RSGAEPTIENNHPNTHSAGSRPHPPEKVPNKEPKDGSPVQPSL | |
| | LSLMKDRRLTLEQVVAIEALTQLSEAPSESSSPSKPEKDEEAH | |
| | QKTASLLNSCKAILHSVRKDLQDPNVQGKGLHHDTVVENGQNR | |
| | TFKSPDSFATNQALIKSQGYPSSPTAEKKGAAGGRAPFDGFEN | |
| | SHPLPIESHNLENCSQVLSCDQNLSSHDPSCQDAPYSQIEEDV | |
| | AAQLTQLASTINHINAEVRNAESTPESLVAKNTKQKHSQEKRM | |
| | VHQKPPSSTQTKPSVPSAKPKKAQKKARATPHANKRKKKPPAR | |
| | SSQENDQKKQEQLAIEYSKMHDIWMSSKFQRFGQSSPRSFPVL | |
| | LRNIPVFNQILKPVTQSKTPSQHNELFPPINQIKFTRNPELAK | |
| | EKVKVEPSDSLPTCQFKTESGGQTFAEPADNSQGQPMVSVNQE | |
| | AHPLPQSPPSNQCANIMAGAAQTQFHLGAQENLVHQIPPPTLP | |
| | GTSPDTLLPDPASILRKGKVLHEDGITVVTEKREAQTSSNGPL | |
| | GPTTDSAQSEFKESIMDLLSKPAKNLIAGLKEQEAAPCDCDGG | |
| | TQKEKGPYYTHLGAGPSVAAVRELMETRFGQKGKAIRIEKIVF | |
| | TGKEGKSSQGCPVAKWVIRRSGPEEKLICLVRERVDHHCSTAV | |
| | IVVLILLWEGIPRLMADRLYKELTENLRSYSGHPTDRRCTLNK | |
| | KRTCTCQGIDPKTCGASFSFGCSWSMYFENGCKEGRSENPRKER | |
| | LAPNYPLHEKQLEKNLQELATVLAPLYKQMAPVAYQNQVEYEE | |
| | VAGDCRLGNEEGRPFSGVTCCMDFCAHSHKDIHNMHNGSTVVC | |
| | TLIRADGRDTNCPEDEQLHVLPLYRLADTDEFGSVEGMKAKIK | |
| | SGAIQVNGPTRKRRLRFTEPVPRCGKRAKMKQNHNKSGSHNTK | |
| | SFSSASSTSHLVKDESTDFCPLQASSAETSTCTYSKTASGGFA | |
| | ETSSILHCTMPSGAHSGANAAAGECTGTVQPAEVAAHPHQSLP | |
| | TADSPVHAEPLTSPSEQLTSNQSNQQLPLLSNSQKLASCQVED | |
| | ERHPEADEPQHPEDDNLPQLDEFWSDSEEIYADPSFGGVAIAP | |
| | IHGSVLIECARKELHATTSLRSPKRGVPFRVSLVFYQHKSLNK | |
| | PNHGFDINKIKCKCKKVTKKKPADRECPDVSPEANLSHQIPSR | |
| | VASTLTRDNVVTVSPYSLTHVAGPYNRWV | |
| Murine | MEQDRTTHAEGTRLSPFLIAPPSPISHTEPLAVKLQNGSPLAE | 5 |
| Tet2 | RPHPEVNGDTKWQSSQSCYGISHMKGSQSSHESPHEDRGYSRC | |
| | LQNGGIKRTVSEPSLSGLHPNKILKLDQKAKGESNIFEESQER | |
| | NHGKSSRQPNVSGLSDNGEPVTSTTQESSGADAFPTRNYNGVE | |
| | IQVLNEQEGEKGRSVTLLKNKIVLMPNGATVSAHSEENTRGEL | |
| | LEKTQCYPDCVSIAVQSTASHVNTPSSQAAIELSHEIPQPSLT | |
| | SAQINFSQTSSLQLPPEPAAMVTKACDADNASKPAIVPGTCPF | |
| | QKAEHQQKSALDIGPSRAENKTIQGSMELFAEEYYPSSDRNLQ | |
| | ASHGSSEQYSKQKETNGAYFRQSSKFPKDSISPTTVTPPSQSL | |
| | LAPRLVLQPPLEGKGALNDVALEEHHDYPNRSNRTLLREGKID | |
| | HQPKTSSSQSLNPSVHTPNPPLMLPEQHQNDCGSPSPEKSRKM | |
| | SEYLMYYLPNHGHSGGLQEHSQYLMGHREQEIPKDANGKQTQG | |
| | SVQAAPGWIELKAPNLHEALHQTKRKDISLHSVLHSQTGPVNQ | |
| | MSSKQSTGNVNMPGGFQRLPYLQKTAQPEQKAQMYQVQVNQGP | |
| | SPGMGDQHLQFQKALYQECIPRTDPSSEAHPQAPSVPQYHFQQ | |
| | RVNPSSDKHLSQQATETQRLSGFLQHTPQTQASQTPASQNSNF | |
| | PQICQQQQQQQLQRKNKEQMPQTFSHLQGSNDKQREGSCFGQI | |
| | KVEESFCVGNQYSKSSNFQTHNNTQGGLEQVQNINKNFPYSKI | |
| | LTPNSSNLQILPSNDTHPACEREQALHPVGSKTSNLQNMQYFP | |
| | NNVTPNQDVHRCFQEQAQKPQQASSLQGLKDRSQGESPAPPAE | |
| | AAQQRYLVHNEAKALPVPEQGGSQTQTPPQKDTQKHAALRWLL | |
| | LQKQEQQQTQQSQPGHNQMLRPIKTEPVSKPSSYRYPLSPPQE | |
| | NMSSRIKQEISSPSRDNGQPKSIIETMEQHLKQFQLKSLCDYK | |
| | ALTLKSQKHVKVPTDIQAAESENHARAAEPQATKSTDCSVLDD | |
| | VSESDTPGEQSQNGKCEGCNPDKDEAPYYTHLGAGPDVAAIRT | |
| | LMEERYGEKGKAIRIEKVIYTGKEGKSSQGCPIAKWVYRRSSE | |
| | EEKLLCLVRVRPNHTCETAVMVIAIMLWDGIPKLLASELYSEL | |
| | TDILGKCGICTNRRCSQNETRNCCCQGENPETCGASFSFGCSW | |
| | SMYYNGCKFARSKKPRKFRLHGAEPKEEERLGSHLQNLATVIA | |
| | PIYKKLAPDAYNNQVEFEHQAPDCCLGLKEGRPESGVTACLDE | |
| | SAHSHRDQQNMPNGSTVVVTLNREDNREVGAKPEDEQFHVLPM | |
| | YIIAPEDEFGSTEGQEKKIRMGSIEVLQSFRRRRVIRIGELPK | |
| | SCKKKAEPKKAKTKKAARKRSSLENCSSRTEKGKSSSHTKLME | |
| | NASHMKQMTAQPQLSGPVIRQPPTLQRHLQQGQRPQQPQPPQP | |
| | QPQTTPQPQPQPQHIMPGNSQSVGSHCSGSTSVYTRQPTPHSP | |
| | YPSSAHTSDIYGDTNHVNFYPTSSHASGSYLNPSNYMNPYLGL | |
| | LNQNNQYAPFPYNGSVPVDNGSPFLGSYSPQAQSRDLHRYPNQ | |
| | DHLTNQNLPPIHTLHQQTFGDSPSKYLSYGNQNMQRDAFTTNS | |
| | TLKPNVHHLATFSPYPTPKMDSHFMGAASRSPYSHPHTDYKTS | |
| | EHHLPSHTIYSYTAAASGSSSSHAFHNKENDNIANGLSRVLPG | |
| | FNHDRTASAQELLYSLTGSSQEKQPEVSGQDAAAVQEIEYWSD | |
| | SEHNFQDPCIGGVAIAPTHGSILIECAKCEVHATTKVNDPDRN | |
| | HPTRISLVLYRHKNLFLPKHCLALWEAKMAEKARKEEECGKNG | |
| | SDHVSQKNHGKQEKREPTGPQEPSYLRFIQSLAENTGSVTTDS | |
| | TVTTSPYAFTQVTGPYNTFV | |
| Murine | MSQFQVPLAVQPDLSGLYDFPQGQVMVGGFQGPGLPMAGSETQ | 6 |
| Tet3 | LRGGGDGRKKRKRCGTCDPCRRLENCGSCTSCTNRRTHQICKL | |
| | RKCEVLKKKAGLLKEVEINAREGTGPWAQGATVKTGSELSPVD | |
| | GPVPGQMDSGPVYHGDSRQLSTSGAPVNGAREPAGPGLLGAAG | |
| | PWRVDQKPDWEAASGPTHAARLEDAHDLVAFSAVAEAVSSYGA | |
| | LSTRLYETFNREMSREAGSNGRGPRPESCSEGSEDLDTLQTAL | |
| | ALARHGMKPPNCTCDGPECPDFLEWLEGKIKSMAMEGGQGRPR | |
| | LPGALPPSEAGLPAPSTRPPLLSSEVPQVPPLEGLPLSQSALS | |
| | IAKEKNISLQTAIAIEALTQLSSALPQPSHSTSQASCPLPEAL | |
| | SPSAPFRSPQSYLRAPSWPVVPPEEHPSFAPDSPAFPPATPRP | |
| | EFSEAWGTDTPPATPRNSWPVPRPSPDPMAELEQLLGSASDYI | |
| | QSVFKRPEALPTKPKVKVEAPSSSPAPVPSPISQREAPLLSSE | |
| | PDTHQKAQTALQQHLHHKRNLFLEQAQDASFPTSTEPQAPGWW | |
| | APPGSPAPRPPDKPPKEKKKKPPTPAGGPVGAEKTTPGIKTSV | |
| | RKPIQIKKSRSRDMQPLFLPVRQIVLEGLKPQASEGQAPLPAQ | |
| | LSVPPPASQGAASQSCATPLTPEPSLALFAPSPSGDSLLPPTQ | |
| | EMRSPSPMVALQSGSTGGPLPPADDKLEELIRQFEAEFGDSFG | |
| | LPGPPSVPIQEPENQSTCLPAPESPFATRSPKKIKIESSGAVT | |
| | VLSTTCFHSEEGGQEATPTKAENPLTPTLSGFLESPLKYLDTP | |
| | TKSLLDTPAKKAQSEFPTCDCVEQIVEKDEGPYYTHLGSGPTV | |
| | ASIRELMEDRYGEKGKAIRIEKVIYTGKEGKSSRGCPIAKWVI | |
| | RRHTLEEKLLCLVRHRAGHHCQNAVIVILILAWEGIPRSLGDT | |
| | LYQELTDTLRKYGNPTSRRCGLNDDRTCACQGKDPNTCGASES | |
| | FGCSWSMYFNGCKYARSKTPRKFRLTGDNPKEEEVLRNSFQDL | |
| | ATEVAPLYKRLAPQAYQNQVTNEDVAIDCRLGLKEGRPFSGVT | |
| | ACMDFCAHAHKDQHNLYNGCTVVCTLTKEDNRCVGQIPEDEQL | |
| | HVLPLYKMASTDEFGSEENQNAKVSSGAIQVLTAFPREVRRLP | |
| | EPAKSCRQRQLEARKAAAEKKKLQKEKLSTPEKIKQEALELAG | |
| | VTTDPGLSLKGGLSQQSLKPSLKVEPQNHESSFKYSGNAVVES | |
| | YSVLGSCRPSDPYSMSSVYSYHSRYAQPGLASVNGFHSKYTLP | |
| | SFGYYGFPSSNPVFPSQFLGPSAWGHGGSGGSFEKKPDLHALH | |
| | NSLNPAYGGAEFAELPGQAVATDNHHPIPHHQQPAYPGPKEYL | |
| | LPKVPQLHPASRDPSPFAQSSSCYNRSIKQEPIDPLTQAESIP | |
| | RDSAKMSRTPLPEASQNGGPSHLWGQYSGGPSMSPKRTNSVGG | |
| | NWGVFPPGESPTIVPDKLNSFGASCLTPSHFPESQWGLFTGEG | |
| | QQSAPHAGARLRGKPWSPCKFGNGTSALTGPSLTEKPWGMGTG | |
| | DENPALKGGPGFQDKLWNPVKVEEGRIPTPGANPLDKAWQAFG | |
| | MPLSSNEKLFGALKSEEKLWDPFSLEEGTAEEPPSKGVVKEEK | |
| | SGPTVEEDEEELWSDSEHNFLDENIGGVAVAPAHCSILIECAR | |
| | RELHATTPLKKPNRCHPTRISLVFYQHKNLNQPNHGLALWEAK | |
| | MKQLAERARQRQEEAARLGLGQQEAKLYGKKRKWGGAMVAEPQ | |
| | HKEKKGAIPTRQALAMPTDSAVTVSSYAYTKVTGPYSRWI | |
| NgTET | MTTFKQQTIKEKETKRKYCIKGTTANLTQTHPNGPVCVNRGEE | 7 |
| | VANTTTLLDSGGGINKKSLLQNLLSKCKTTFQQSFTNANITLK | |
| | DEKWLKNVRTAYFVCDHDGSVELAYLPNVLPKELVEEFTEKFE | |
| | SIQTGRKKDTGYSGILDNSMPFNYVTADLSQELGQYLSEIVNP | |
| | QINYYISKLLTCVSSRTINYLVSLNDSYYALNNCLYPSTAENS | |
| | LKPSNDGHRIRKPHKDNLDITPSSLFYFGNFQNTEGYLELTDK | |
| | NCKVFVQPGDVLFFKGNEYKHVVANITSGWRIGLVYFAHKGSK | |
| | TKPYYEDTQKNSLKIHKETK | |

In some embodiments of the present disclosure, the TET used herein is a variant of a naturally occurring TET comprising one or more mutations. In some embodiments, the TET used herein is a truncated variant of a naturally occurring TET. The truncation can be located outside the core catalytic region or outside the conserved double-stranded-helix (DSBH) domain of TET.

The TET used herein can, for example, comprise, or consist of, an amino acid sequence having at least 50% sequence identity to an amino acid sequence of any of the TET proteins disclosed herein (e.g. SEQ ID NO: 1-7). In some embodiments, the TET protein comprises, or consists of, an amino acid sequence having, or having about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, 100%, or a range between any two of these values, sequence identity to an amino acid sequence of any one of SEQ ID NO: 1-7. In some embodiments, the TET protein comprises, or consists of, an amino acid sequence having at least, or at least about, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 97.5%, 98%, 98.5%, 99%, 99.5%, sequence identity to an amino acid sequence of any one of SEQ ID NO: 1-7.

The TET protein or variants thereof can, for example, comprise, or consists of, an amino acid sequence having, or having about, one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty, or a range between any two of these values, mismatch compared to an amino acid sequence of any of the TET proteins disclosed herein (e.g., TET proteins having an amino acid sequence of any one of SEQ ID NOs: 1-7). In some embodiments, the TET protein or variants thereof comprises, or consists of, an amino acid sequence having at most, or having at most about, one, two, three, four, five, six, seven, eight, nine, ten, eleven, twelve, thirteen, fourteen, fifteen, sixteen, seventeen, eighteen, nineteen, twenty, twenty-two, twenty-three, twenty-four, twenty-five, twenty-six, twenty-seven, twenty-eight, twenty-nine, thirty mismatches compared to an amino acid sequence selected from the group consisting of SEQ ID NOs: 1-7.

The TET enzymes used herein can be a wild type protein naturally occurring such as SEQ ID NO: 1-7. The TET enzymes used herein can also be engineered enzymes that are modified using protein engineering methods such as directed evolution. The term “directed evolution” is a method used in protein engineering that mimics the process of natural selection to steer proteins or nucleic acids toward a desired activity and selectivity. Therefore, the TET variant herein described can be tuned by directed evolution to enhance its non-natural carbene-insertion capability while inhibiting its natural oxidation reaction capability.

In some embodiments, the TET variants can have an enhanced carbene-insertion activity of at least about 1.5 to 2,000 fold, for example, at least about 1.5, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,550, 1,600, 1,650, 1,700, 1,750, 1,800, 1,850, 1,900, 1,950, 2,000, or more fold compared to the corresponding wild-type TET protein.

Variations in the TET enzymes can be introduced into a target gene naturally encoding a TET enzyme using standard cloning techniques (e.g. site-directed mutagenesis, site-saturated mutagenesis) or by gene synthesis to produce the TET enzymes.

The TET enzymes and variants thereof used herein can be extracted or purified from the cells where they are present. The TET enzymes and variants thereof can also be recombinantly expressed and then isolated and/or purified. The TET enzymes and variants thereof can also be expressed in one or more host cells and carried out the reactions disclosed herein within the host cells in vivo or ex vivo.

The TET enzymes and variants thereof can be expressed in cells such as bacterial cells, archaeal cells, yeast cells, fungal cells, insect cells, plant cells, or mammalian cells using an expression vector under the control of an inducible promoter or a constitutive promoter. The expression vector comprising a nucleic acid sequence that encodes the TET enzymes or variants can be a viral vector, a plasmid, a phage, a phagemid, a cosmid, a fosmid, a bacteriophage (e.g., a bacteriophage P1-derived vector (PAC)), a baculovirus vector, a yeast plasmid, or an artificial chromosome (e.g., bacterial artificial chromosome (BAC), a yeast artificial chromosome (YAC), a mammalian artificial chromosome (MAC), and human artificial chromosome (HAC)). Expression vectors can include chromosomal, non-chromosomal, and synthetic DNA sequences. Equivalent expression vectors to those described herein are known in the art and will be apparent to a skilled person in the art.

In embodiments herein described, the TET or variants thereof disclosed herein carry out anon-natural reaction that is diverted from its natural oxidation reaction. The non-natural reaction results in a carbene-insertion in the 5-methyl moiety of 5mC or the 5-hydroxymethyl moiety of 5hmC, thereby generating a modified nucleic acid base that can form a hydrogen bond with adenine (A) and thus read directly as or copied to Thymine (T) via amplification.

FIG. 4 illustrates a non-limiting example of a chemoenzymatic carbene-modification of MeC by TET of SEQ ID NO: 2. The left panel of FIG. 4 shows a crystal structure of the iron-containing active site of TET (SEQ ID NO: 2). The top row of the right panel illustrates a natural TET-mediated oxidation of MeC. The bottom row of the right panel illustrates a modified, non-natural TET-mediated carbene-insertion followed by spontaneous cyclization and tautomerization to generate a modified nucleic acid adduct. In the natural reaction (top row, right panel), the MeC is converted into a 5-carboxy C (HO-MeC). In the non-natural reaction (bottom row, right panel), the carbene-mediated modification, cyclization and tautomerization generates a new Watson Crick hydrogen bonding face that reads directly as or is copied to T via amplification. In some embodiments, the tautomerization can be tuned by the nature of the substituent group (R), for example an electron-withdrawing group.

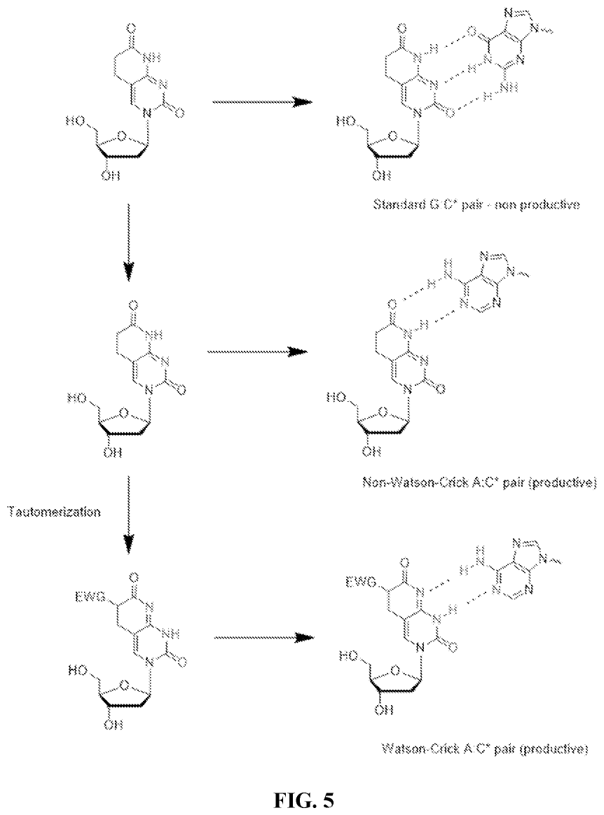

FIG. 5 illustrates a non-limiting example of the cyclization and tautomerization of the cyclized product following the carbene-modification of MeC in order to alter the Watson-Crick hydrogen bonding face of the modified-MeC base.

Methods for Identifying 5-Methylcytosine (5mC) and/or 5-Hydroxymethylcytosine (5hmC) in a Target Nucleic Acid

Provided herein includes a method for identifying 5-methylcytosine (5mC), 5-hydroxymethylcytosine (5hmC), or both in a target nucleic acid. The method, in some embodiments, includes (a) providing a nucleic acid sample comprising a target nucleic acid suspected of comprising, or comprising, one or more 5-methylcytosine (5mC) or 5-hydroxymethylcytosine (5hmC), (b) performing a TET-mediated carbene insertion on the 5-methyl moiety of the 5mC or the 5-hydroxymethyl moiety of 5hmC in the target nucleic acid to generate a modified target nucleic acid, and (c) determining the sequence of the modified target nucleic acid, wherein a cytosine (C) to (T) transition in the sequence of the modified target nucleic acid compared to the sequence of the target nucleic acid indicates a 5mC or 5hmC in the target nucleic acid.

In some embodiments disclosed herein, the step of performing a TET-mediated carbene insertion in the 5-methyl moiety of the 5mC or the 5-hydroxymethyl moiety of 5hmC in a target nucleic acid comprises contacting the target nucleic acid with a TET or a variant thereof, thereby producing a C—H insertion on the 5-methyl moiety of the 5mC or the 5-hydroxymethyl moiety of 5hmC.

The production of a C—H insertion on the 5-methyl moiety of the 5mC or the 5-hydroxymethyl moiety of 5hmC in a target nucleic acid can be accomplished by using the reaction mixtures disclosed herein comprising a TET enzyme or variants thereof and a carbene precursor.

The reactions can be conducted under conditions sufficient to catalyze a carbene insertion in a nucleic acid comprising 5-methylcytosine (5mC), 5-hydroxymethylcytosine (5hmC) or both. For example, the reactions can be conducted at any suitable temperature. In general, the reactions are conducted at a temperature of from about 0° C. to about 40° C. The reactions can be conducted, for example, at about 25° C. or about 37° C. In certain embodiments, high stereoselectivity can be achieved by conducting the reaction at a temperature less than 25° C. (e.g., around 20° C., 10° C. or 4° C.) without reducing the total turnover number of the enzyme catalyst.

The reactions can be conducted at any suitable pH. In general, the reactions are conducted at a pH of from about 6 to about 10. The reactions can be conducted, for example, at a pH of from about 6.5 to about 9 (e.g., about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0).

The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 18 hours, or about 24 hours, or about 48 hours, or about 72 hours. In some embodiments, the reactions are conducted for a period of time ranging from about 6 hours to about 24 hours (e.g., about 6, 7, 8, 9, 10, 11, 12, 13, 14, 15, 16, 17, 18, 19, 20 21, 22, 23, or 24 hours).

Contacting the target nucleic acid with a TET or a variant thereof can be performed under aerobic conditions or anaerobic conditions.

In some embodiments, the contacting are performed under anaerobic conditions, thereby diverting the natural TET-mediate oxidation of MeC to HO-MeC into a non-natural carbene-insertion reaction in the 5-methyl moiety of 5-mC or the 5-hydroxymethyl moiety of 5-hmC by removing oxygen. Reactions can be conducted under an inert atmosphere, such as a nitrogen atmosphere or argon atmosphere, by sparging a reaction mixture with an inert gas such as nitrogen or argon.

In some embodiments, the contacting are performed under aerobic conditions. The reaction can be conducted in the presence of a non-reducing acid or a salt thereof to divert the natural TET-mediate oxidation of MeC to HO-MeC into a non-natural carbene-insertion reaction in the 5-methyl moiety of 5-mC or the 5-hydroxymethyl moiety of 5-hmC.

Upon a carbene-insertion reaction, 5mC, 5hmC or both are converted into a modified nucleic acid adduct, which, upon spontaneous cyclization and tautomerization, can hybridize like thymine, while the methylated cytosine in the unmodified target nucleic acid hybridizes like cytosine. In some embodiments, the tautomerization can be tuned by the nature of the substituent group (R), for example an electron-withdrawing group. The modified target nucleic acid contains a modified nucleic acid adduct at positions wherein one or more of 5mC, 5hmC or both were present in the unmodified target nucleic acid. The modified nucleic acid adduct can be detected directly or replicated by known methods wherein the modified nucleic acid adduct is converted to T. This difference in hybridization properties can be detected by comparing the sequence of the unmodified target nucleic acid with the sequence of the modified target nucleic acid. Thus, the method disclosed herein identifies the location of 5mC and/or 5hmC by identifying the presence of a mismatch (a C to T transition).

The methods disclosed herein can perform nucleic acid methylation and hydroxymethylation analysis under a mild, nontoxic and bisulfite-free condition using a one-step chemoenzymatic modification of methylated cytosines by directly converting methylated cytosines into a modified nucleic acid adduct that can be “read” as T by common polymerases, without affecting unmethylated cytosines while avoiding multiple step chemical reactions associated with EM-Seq and TAPS which commonly lead to incomplete conversion.

### Nucleic Acid Sample and Target Nucleic Acid

The present disclosure provides methods and reaction mixtures for identifying 5-methylcytosine (5mC) or 5-hydroxymethylcytosine (5hmC) in a target nucleic acid.

In some embodiments disclosed herein, the target nucleic acid is DNA, for example genomic DNA. In other embodiments, the target nucleic acid is RNA. Likewise the nucleic acid sample that comprises the target nucleic acid may be a DNA sample and/or an RNA sample.

The target nucleic acid can be any nucleic acid having cytosine modifications (e.g., 5mC, 5hmC). The target nucleic acid can be a single nucleic acid molecule in a nucleic acid sample, or may be the entire population of nucleic acid molecules in a sample or a subset thereof. The target nucleic acid can be the native nucleic acid from the source (e.g., cell, tissue samples) or can pre-converted into a high-throughput sequencing-ready form, for example by amplification, fragmentation, repair and ligation with adaptors for sequencing. Thus, target nucleic acids can comprise a plurality of nucleic acid sequences such that the methods described herein may be used to generate a library of target nucleic acid sequences that can be analyzed individually (e.g., by determining the sequence of individual targets) or in a group (e.g., by high-throughput or next generation sequencing methods).

A nucleic acid sample can be obtained from any organism of interest from the Monera (bacteria), Protista, Fungi, Plantae, and Animalia Kingdoms. The nucleic acid sample can be a mammalian sample, and particularly a human sample.

In embodiments disclosed herein, the nucleic acid sample may be extracted or derived from a single cell, a collection of cells, cell lines, a body fluid, a tissue sample, an organ, and an organelle.

Nucleic acid samples used herein may be obtained from any source including a clinical sample and a derivative thereof, an environmental sample and a derivative thereof, an agricultural sample and a derivative thereof, and a combination thereof. The nucleic acid sample can also be a water sample and a derivative thereof, a produce sample and a derivative thereof, a biological sample and a derivative thereof, or bodily fluids and a derivative thereof including, but not limited to, blood, urine, serum, lymph, saliva, anal, and vaginal secretions, perspiration and semen of any organism.

The methods and reaction mixtures herein described utilize a mild, bisulfite-free, one-step chemoenzymatic reaction that avoids multiple step chemical reactions associated with existing methods such as EM-Seq and TAPS and the substantial degradation associated with methods such as bisulfate sequencing. Thus, the methods disclosed herein are useful in analysis of low-input samples, such as circulating cell-free DNA, in single-cell analysis and low-input RNA-seq.

### Amplifying the Modified Target Nucleic Acid

The methods of the present disclosure may also comprise the step of amplifying the modified target nucleic acid to increase the copy number of the modified target nucleic acid by methods known in the art.

Any form of amplification can be used herein including, but not limited to, transcription mediated amplification, nucleic acid sequence-based amplification, signal mediated amplification of RNA technology, strand displacement amplification, rolling circle amplification, loop-mediated isothermal amplification of DNA, isothermal multiple displacement amplification, helicase-dependent amplification, single primer isothermal amplification, circular helicase-dependent amplification, and others identifiable to a person skilled in the art.

When the modified target nucleic acid is DNA, the copy number can be increased by, for example, PCR, cloning, and primer extension. The copy number of individual target DNAs can be amplified by PCR using primers specific for a particular target DNA sequence. Alternatively, a plurality of different modified target DNA sequences can be amplified by cloning into a DNA vector by standard techniques.

Some embodiments disclosed herein include preparing amplified libraries of target nucleic acids. The copy number of a plurality of different modified target nucleic acid sequences can be increased by PCR to generate a library for next generation sequencing where, e.g., adapter sequence has been ligated to the target nucleic acid or to the modified target nucleic acid and PCR is performed using primers complimentary to the adapter sequence. Library preparation can be accomplished by random fragmentation of DNA, followed by in vitro ligation of common adaptor sequences as will be understood by a person skilled in the art.

### Determining the Sequence of the Modified Target Nucleic Acid

In embodiments disclosed herein, the method comprises the step of determining the sequence of the modified target nucleic acid, wherein a cytosine (C) to (T) transition in the sequence of the modified target nucleic acid compared to the sequence of the target nucleic acid indicates a 5mC and/or 5hmC in the target nucleic acid.

The modified target nucleic acid contains a modified nucleic acid adduct at positions wherein one or more of 5mC, 5hmC or both were present in the unmodified target nucleic acid. The modified nucleic acid adduct acts as a T in nucleic acid replication and sequencing methods. Thus, the cytosine modifications can be detected by any direct or indirect method that identifies a C to T transition know in the art.

The methods and reaction mixtures described herein can be used in conjunction with a variety of sequencing methods, for example next generation sequencing methods (including but not limited to sequencing-by-synthesis (SBS) technologies).

Sequencing-by-synthesis generally involves the enzymatic extension of a nascent primer through the iterative addition of nucleotides against a template strand to which the primer is hybridized. Briefly, SBS can be initiated by contacting target nucleic acids, attached to sites in a flow cell, with one or more labeled nucleotides, DNA polymerase, etc. Those sites where a primer is extended using the target nucleic acid as template will incorporate a labeled nucleotide that can be detected. Detection can include scanning using an apparatus or method set forth herein. Optionally, the labeled nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the vessel (before or after detection occurs). Washes can be carried out between the various delivery steps. The cycle can be performed n times to extend the primer by n nucleotides, thereby detecting a sequence of length n. Exemplary SBS procedures, reagents and detection components that can be readily adapted for use with the methods, compositions, systems and apparatus disclosed herein are described, for example, in Bentley et al., Nature 456:53-59 (2008), WO 04/018497; WO 91/06678; WO 07/123744; U.S. Pat. Nos. 7,057,026; 7,329,492; 7,211,414; 7,315,019 and 7,405,281, and US Pat. App. Pub. No. 2008/0108082 A1, each of which is incorporated herein by reference. Also useful are SBS methods that are commercially available from Illumina, Inc. (San Diego, Calif.). One or more reagents used in an SBS process can optionally be delivered via a mixed-phase fluid (e.g. a fluid foam, fluid slurry or fluid emulsion), contacted with a mixed-phase fluid, and/or removed by a mixed-phase fluid. A mixed-phase fluid can be removed from a flow cell for detection during an SBS process.

Some embodiments of the sequencing-by-synthesis technologies use pyrosequencing which detects the release of inorganic pyrophosphate as particular nucleotides incorporated into the nascent strand as described, for example, in Ronaghi et al., Analytical Biochemistry 242 (1): 84-9 (1996); Ronaghi, M. Genome Res. 11 (1): 3-11(2001); Ronaghi et al., Science 281 (5375): 363(1998); U.S. Pat. Nos. 6,210,891; 6,258,568 and 6,274,320, each of which is incorporated by reference in its entirety.

Some embodiments of the sequencing technology described herein can utilize sequencing by ligation techniques which utilize DNA ligase to incorporate nucleotides and identify the incorporation of such nucleotides. Exemplary SBS systems and methods which can be utilized with the methods disclosed herein are described in U.S. Pat. Nos. 6,969,488, 6,172,218, and 6,306,597, each of which is incorporated by reference in its entirety.

Some embodiments of the sequencing technology described herein can include techniques such as next-next technologies. One example can include nanopore sequencing techniques as described, for example, in Deamer & Akeson “Nanopores and nucleic acids: prospects for ultrarapid sequencing. “Trends Biotechnol. 18, 147-151 (2000); Deamer and Branton, “Characterization of nucleic acids by nanopore analysis”. Acc. Chem. Res. 35: 817-825 (2002); Li et al., “DNA molecules and configurations in a solid-state nanopore microscope “Nat. Mater. 2: 611-615 (2003), each of which is incorporated by reference in its entirety. In such embodiments, the target nucleic acid passes through a nanopore. The nanopore can be a synthetic pore or biological membrane protein. As the target nucleic acid passes through the nanopore, each base-pair can be identified by measuring fluctuations in the electrical conductance of the pore.

Some embodiments of the sequencing technology described herein can utilize methods involving the real-time monitoring of DNA polymerase activity. Nucleotide incorporations can be detected through fluorescence resonance energy transfer (FRET) interactions between a fluorophore-hearing polymerase and γ-phosphate-labeled nucleotides as described, for example, in U.S. Pat. Nos. 7,329,492 and 7,211,414 or nucleotide incorporations can be detected with zero-mode waveguides as described, for example, in U.S. Pat. No. 7,315,019 and using fluorescent nucleotide analogs and engineered polymerases as described, for example, in U.S. Pat. No. 7,405,281 and U.S. Patent Application Publication No. 2008/0108082, each of which is incorporated by reference in its entirety. In one example, single molecule, real-time (SMRT) DNA sequencing technology can be utilized with the methods described herein.

It will be appreciated by one of skill in the art that other known sequencing processes can be easily implemented for use with the methods, compositions, kits and systems described herein.

### Kits

Provided herein also includes kits for identifying 5-methylcytosine (5mC), 5-hydroxymethylcytosine (5hmC), or both in a target nucleic acid. In some embodiments herein disclosed, the kits can include one or more of the TET enzymes or variants thereof described above. For example, the TET enzyme can be selected from the group consisting of human TET1, TET2, TET3, and variants thereof, murine Tet1, Tet2, Tet3, and variants thereof, Naegleria TET (NgTET) and variants thereof, Coprinopsis cinerea (CcTET) and variants thereof, and a combination thereof. The TET enzyme can be, for example, a prokaryotic TET enzyme or a eukaryotic TET enzyme. In some embodiments, the TET enzyme is a viral TET enzyme, for example a bacteriophage TET. Non-limiting examples of phase-encoded TET are described in, for example, Burket et al. PNAS Jun. 29, 2021 118 (26) e2026742118, the content of which is hereby expressly incorporated by references.

The kits can also include one or more nucleic acid molecules comprising a nucleotide sequence encoding a TET enzyme or variants thereof described above. In some embodiments, the nucleic acid molecule is an expression vector. The expression vector comprising a nucleic acid sequence that encodes the TET enzymes or variants described herein can be a viral vector, a plasmid, a phage, a phagemid, a cosmid, a fosmid, a bacteriophage (e.g., a bacteriophage P1-derived vector (PAC)), a baculovirus vector, a yeast plasmid, or an artificial chromosome (e.g., bacterial artificial chromosome (BAC), a yeast artificial chromosome (YAC), a mammalian artificial chromosome (MAC), and human artificial chromosome (HAC)). In some embodiments, the nucleotide sequence is operably linked to a transcriptional control element such as promoters, enhancers, and post-transcriptional and post-translational regulatory sequences that are compatible with the expression of TET proteins as will be understood by a person skilled in the art.

The kits comprise a carbene precursor herein disclosed. The carbene precursor can be one or more of diazo reagents, diazirine reagents, hydrozone reagents, and a combination thereof as described herein.

The kits can include a non-reducing acid or a salt thereof described above, selected from the group consisting of acetic acid, dichloroacetic acid, fluoroacetic acid, chloroacetic acid, citric acid, ascorbic acid, benzoic acid, and a combination thereof.

The kits can include reagents for isolating DNA or RNA, reagents, buffers, and substrate solutions for amplifying and sequencing the nucleic acid, and additional reagents suitable for the detection and purification of the modified target nucleic acid in downstream applications, as known to one of skill in the art. The kit can, for example, include the compositions in separate containers. The kits can also include instructions and one or more additional reagents for performing the methods herein disclosed.

### EXAMPLES

Some aspects of the embodiments discussed above are disclosed in further detail in the following examples, which are not in any way intended to limit the scope of the present disclosure.

### Example 1

### Carbene and Nitrene Insertion Reactions Carried Out by Heme-Bound Proteins and Non-Heme Iron Oxidases

This example illustrates exemplary chemical reactions carried out by heme-bound proteins and non-heme iron oxidases such as TET.

TET is a non-heme iron oxygenase that carries out oxidation of MeC using an enzyme bound iron catalyst, a small molecule cofactor (alpha-ketoglutarate, aKG) for iron reduction, and molecular oxygen as the oxygenation source. The key feature of this family of enzymes is the iron center, which is the active catalyst for these enzymes. Similar chemistry is observed in other enzymes, including heme-containing proteins such as globins and cytochrome P450s (FIG. 2 and FIG. 3.)

FIG. 2 illustrates wild type catalysis (monooxygenation), carbene insertion (C—C bond formation) and nitrene insertion (C—N bond formation) reactions carried out heme bound proteins such as cytochrome P450.

FIG. 3 illustrates wild type catalysis (monooxygenation), carbene insertion (C—C bond formation) and nitrene insertion (C—N bond formation) reactions carried out by non-heme iron oxidases such as TET.

In nature, both heme proteins and non-heme iron oxidases are capable of oxidizing C—H bonds to alcohols (C—OH bonds) using molecular oxygen as an oxygen atom donor/oxidant. This chemistry occurs via a highly reactive iron-oxo intermediate shown in FIGS. 2 and 3.

Previous studies have shown that using a heme enzyme, replacing oxygen with a synthetic diazo-acetate reagent enable access to a synthetic iron-carbon intermediate (iron carbenoid) that is similar in structure to the wild type iron-oxo intermediate. Access to this intermediate allows the enzyme to insert a carbon center into the C—H bond creating a new carbon-carbon (C—C) bond (see middle panel, FIGS. 2 and 3) (Review, Nature, 2020, DOI: 10.1038/s41929-019-0385-5). Similarly, previous studies also demonstrated that these same enzymes can carry out nitrogen insertion to generate new carbon-nitrogen (C—N) bonds (Angew. Chem. Int. Ed. 2013, DOI 10.1002/anie.201304401). This chemistry has been adapted to the activation of olefins (Science, 2013, DOI: 10.1126/science.1231434), aliphatic C—H bonds (Nature, 2018, DOI: 10.1038/s41586-018-0808-5), benzylic and allylic C—H bonds (JACS, 2020 DOI: 10.1021/acscatal.0c01888), among other bonds. It is also noted that MeC oxidation is carried out on a benzylic-like C—H bond. Additional studies also show that non-heme iron oxidases, homologous to TET, also carry out these chemistries (JACS, 2019 DOI: 10.1021/jacs.9b11608). The related publications herein mentioned are incorporated by reference in their entirety.

As described above, it is expected that a non-heme iron oxidase mediated chemoenzymatic reaction can be used to directly convert methylated cytosine into a novel nucleic acid that can be readout by DNA sequencing.

### Example 2

### A Non-Natural Chemoenzymatic Carbene-Modification of MeC by TET

This example illustrates a non-natural TET-mediated carbene-insertion to directly convert MeC (5mC and/or 5hmC) into a novel DNA base that can be readout by DNA sequencing. This approach is summarized in FIG. 4.

FIG. 4 illustrates a chemoenzymatic carbene-modification of MeC by TET. The left panel of FIG. 4 shows a crystal structure of the iron-containing active site of TET (SEQ ID NO: 1). The top row of the right panel illustrates a natural TET-mediated oxidation of MeC. The bottom row of the right panel illustrates a modified, non-natural TET-mediated carbene-insertion followed by spontaneous cyclization and tautomerization to generate a novel sequenceable base. In the natural reaction (top row, right panel), the MeC is converted into a 5-carboxy C (HO-MeC). In the non-natural reaction (bottom row, right panel), the carbene-mediated modification, cyclization and tautomerization generates a new Watson Crick hydrogen bonding face that reads directly as or is copied to T via PCR.

FIG. 5 illustrates the cyclization and tautomerization of the cyclized product following the carbene-modification of MeC in order to alter the Watson-Crick hydrogen bonding face of the modified-MeC base.

The approach described herein diverts the natural TET-mediate oxidation of MeC to HO-MeC into a non-natural carbene-insertion reaction in the 5-methyl moiety of 5-mC or the 5-hydroxymethyl moiety of 5-hmC. To divert this chemistry, oxygen can be replaced with a synthetic diazoacetate ester reagent. The diazoacetate can generate a new carbon-carbon bond on the 5-methyl moiety of 5-mC or the 5-hydroxymethyl moiety of 5-hmC (See FIG. 4, Right, bottom).

Upon carbene-insertion, the newly added ester group is now located in proximity to the MeC exocyclic amine and this proximity will enforce spontaneous cyclization product that can tautomerize to generate a new base adduct with an altered Watson-Crick hydrogen bonding face that now resembles T. This face will read out as T via direct sequencing, or will be copied as T after amplification via PCR or ExAMP clustering.

### Example 3

### Diversion of a Natural TET-Mediated Oxidation into a Non-Natural TET-Mediated Carbene-Insertion of MeC

Since TET carried out both oxygen insertion and carbon insertion, in order to enforce the non-natural carbene-insertion reaction and inhibit the natural oxidation reaction, the reaction can be carried out under anaerobic condition by removing oxygen from the system. Alternatively, even in the presence of oxygen the carbene-insertion reaction can also be carried out by replacing the cofactor alpha-ketoglutarate of TET with a non-reducing acid such as acetic acid.

Directed evolution can also be used to improve the activity of the TET enzyme in catalyzing this non-natural reaction.

The yield for spontaneous cyclization depends on the nature of the diazoester used and particularly the leaving group that is displaced by the cyclization reaction. This leaving group can be tuned by standard synthetic organic chemistry to enforce the cyclization reaction.

Tautomerization (FIG. 5) can also be enforced via the addition of electron withdrawing groups on the diazo acetate substrate and this effect can be tuned via synthetic chemistry. Nature of hydrogen bonding observed by the tautomerized base can be determined empirically and via optimization by altering the nature of the diazoacetate.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for identifying 5-methylcytosine (5mC), 5-hydroxymethylcytosine (5hmC), or both in a target nucleic acid, comprising;
(a) providing a nucleic acid sample comprising a target nucleic acid suspected of comprising, or comprising, one or more 5-methylcytosine (5mC) or 5-hydroxymethylcytosine (5hmC);
(b) performing a ten eleven translocation enzyme (TET)-mediated carbene insertion on the 5-methyl moiety of the 5mC or the 5-hydroxymethyl moiety of 5hmC in the target nucleic acid to generate a modified target nucleic acid; and
(c) determining the sequence of the modified target nucleic acid;
wherein a cytosine (C) to (T) transition in the sequence of the modified target nucleic acid compared to the sequence of the target nucleic acid indicates a 5mC or 5hmC in the target nucleic acid.

2. The method of claim 1, wherein performing a TET-mediated carbene insertion on the 5-methyl moiety of the 5mC or the 5-hydroxymethyl moiety of 5hmC comprises
contacting the target nucleic acid with a TET or a variant thereof, thereby producing a C—H insertion on the 5-methyl moiety of the 5mC or the 5-hydroxymethyl moiety of 5hmC.

3. The method of claim 1, wherein the TET-mediated carbene insertion comprises converting the 5mC or 5hmC into a modified nucleic acid adduct capable of forming a hydrogen bond with adenine (A).

4. The method of claim 1, wherein the TET-mediated carbene insertion is performed in the presence of a carbene precursor.

5. The method of claim 4, wherein the carbene precursor has a structure of Formula I: 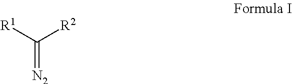
wherein
R¹ is selected from the group consisting of H, —C(O)OR^{1a}, —C(O)R^{1a}, —C(O)N(R^{1b})₂, —SO₂R^{1a}, —SO₂OR¹, —P(O)(OR^{1a})₂, —NO₂, —CN, C₁₋₁₈ alkyl, C₂₋₁₈ alkenyl, C₂₋₁₈ alkynyl, 2- to 18-membered heteroalkyl, C₁₋₁₈ haloalkyl, C₁₋₁₈ alkoxy, C₃₋₁₀ cycloalkyl, C₆₋₁₀ aryl, 3- to 10-membered heterocyclyl, and 5- to 10-membered heteroaryl;
each R^{1a} is independently selected from the group consisting of H, C₁₋₁₈ alkyl, C₂₋₁₈ alkenyl, C₂₋₁₈ alkynyl, C₆₋₁₀ aryl, 3- to 10-membered heterocyclyl, and 5- to 10-membered heteroaryl;
each R^{1b} is independently selected from the group consisting of H, C₁₋₁₈ alkyl, C₂-18 alkenyl, C-₁₈ alkynyl, and C₁₋₁₈ alkoxy;
R² is an electron-withdrawing group selected from the group consisting of —C(O)OR^{2a}, —C(O)R^{2a}, —C(O)N(R^{2b})₂, —SO₂R^{2a}, —SO₂OR^{2a}, —P(O)(OR^{2a})₂, —NO₂, and —CN;
each R^{2a} is independently selected from the group consisting of H, C₁₋₁₈ alkyl, C₂₋₁₈ alkenyl, C₂₋₁₈ alkynyl, C₆₋₁₀ aryl, 3- to 10-membered heterocyclyl, and 5- to 10-membered heteroaryl;
each R^{2b} is independently selected from the group consisting of H, C₁₋₁₈ alkyl, C₂₋₁₈ alkenyl, C₂₋₁₈ alkynyl, and C₁₋₈ alkoxy; and
R¹ and R² are optionally and independently substituted; or
R¹ and R² are taken together to form C₃₋₁₀ cycloalkyl, C₆₋₁₀ aryl, 3- to 10-membered heterocyclyl, and 5- to 10-membered heteroaryl, each of which is optionally substituted.

6. The method of claim 4, wherein the carbene precursor has a structure of Formula I: 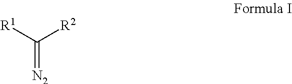
wherein
R¹ is selected from the group consisting of H, —C(O)OR^{1a}, —C(O)R^{1a}, —C(O)N(R^{1b})₂, —SO₂R^{1a}, —SO₂OR^{1a}, —P(O)(OR¹ª)₂, —NO₂, —CN, C₁₋₁₈ alkyl, 2- to 18-membered heteroalkyl, C₁₋₁₈ haloalkyl, C₁₋₁₈ alkoxy, C₃₋₁₀ cycloalkyl, C₆₋₁₀ aryl, 3- to 10-membered heterocyclyl, and 5- to 10-membered heteroaryl;
each R^{1a} is independently C₁₋₈ alkyl;
each R^{1b} is independently selected from the group consisting of H, C₁₋₈ alkyl, and C₁₋₈ alkoxy;
R² is an electron-withdrawing group selected from the group consisting of —C(O)OR^{2a}, —C(O)R^{2a}, —C(O)N(R²⁶)₂, —SO₂R^{2a}, —SO₂OR^{2a}, —P(O)(OR^{2a})₂, —NO₂, and —CN;
each R^{2a} is independently C₁₋₈ alkyl;
each R^{2b} is independently selected from the group consisting of H, C₁₋₈ alkyl, and C₁₋₈ alkoxy; and
R¹ and R² are optionally and independently substituted; or
R¹ and R² are taken together to form C₃₋₁₀ cycloalkyl, C₆₋₁₀ aryl, 3- to 10-membered heterocyclyl, and 5- to 10-membered heteroaryl, each of which is optionally substituted.

7. The method of claim 4, wherein the carbene precursor has a structure of Formula I: 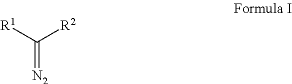
wherein
R¹ is independently selected from the group consisting of H, —C(O)OR^{1a}, —C(O)R^{1a}, —SO₂R^{1a}, —SO₂OR^{1a}, substituted C₁₋₁₈ alkyl, 2- to 18-membered heteroalkyl, C₁₋₁₈ alkoxy, C₃₋₁₀ cycloalkyl, C₁₋₁₈ fluoroalkyl, substituted C₆₋₁₀ aryl, and substituted 5- to 10-membered heteroaryl;
R^{1a} is C₁₋₈ alkyl;
R² is selected from the group consisting of —C(O)OR^{2a}, —C(O)R^{2a}, —SO₂R^{2a}, and —SO₂OR^{2a}; and
R^{2a} is C₁₋₈ alkyl; or
R¹ and R² are optionally taken together to form C₃₋₁₀ cycloalkyl, C₆₋₁₀ aryl, 3- to 10-membered heterocyclyl, and 5- to 10-membered heteroaryl, each of which is optionally substituted.

8. The method of claim 4, wherein the carbene precursor is selected from the group consisting of diazo reagents, diazirine reagents, hydrozone reagents, and a combination thereof.

9. The method of claim 4, wherein the carbene precursor is selected from the group consisting of

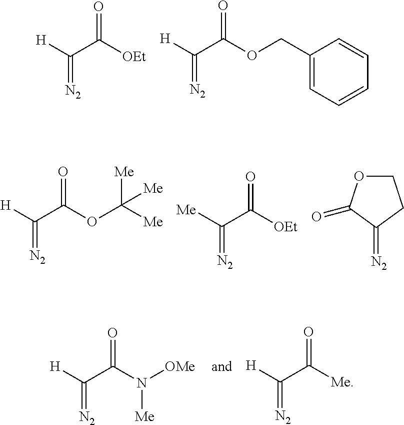

10. The method of claim 4, wherein the carbene precursor is diazoacetate ester.

11. The method of claim 1, wherein the TET is selected from the group consisting of human TET1, TET2, TET3, and variants thereof; murine Tet1, Tet2, Tet3, and variants thereof; Naegleria TET (NgTET) and variants thereof; Coprinopsis cinerea (CcTET) and variants thereof; and a combination thereof.

12. The method of claim 1, wherein the TET is TET1 or ngTET.

13. (canceled)

14. The method of claim 1, wherein performing a TET-mediated carbene insertion on the 5-methyl moiety of the 5mC or the 5-hydroxymethyl moiety of 5hmC is
(a) under an anaerobic condition;
(b) in the presence of a non-reducing acid or a salt thereof; or
(c) combination thereof.

15-16. (canceled)

17. The method of claim 14, wherein the non-reducing acid is selected from the group consisting of acetic acid, n-oxalylglycine, dichloroacetic acid, fluoroacetic acid, chloroacetic acid, citric acid, ascorbic acid, benzoic acid, and a combination thereof.

18. (canceled)

19. The method of claim 1, wherein the target nucleic acid comprises at least one 5mC.

20. The method of claim 1, wherein the target nucleic acid is DNA.

21. The method of claim 1, wherein the target nucleic acid is mammalian genomic DNA or human genomic DNA.

22. (canceled)

23. The method of claim 1, wherein the target nucleic acid is RNA.

24. The method of claim 1, comprising amplifying the modified target nucleic acid after (b) and before (c).

25. The method of claim 1, wherein the nucleic acid sample is selected from the group consisting of a clinical sample and a derivative thereof, an environmental sample and a derivative thereof, an agricultural sample and a derivative thereof, and a combination thereof.

26. The method of claim 1, wherein the method does not comprise
(a) formation of one or more of carboxy cytosine, dihydrouracil and uracil′
(b) conversion of 5mC to carboxy cytosine;
(c) a deamination reaction by a cytidine deaminase, and optionally the cytidine deaminase is an APOBEC;
(d). chemical reduction by a borane reagent; or
(e) use of a borane reagent.

27-30. (canceled)

31. A reaction mixture for performing a ten eleven translocation enzyme (TET)-mediated carbene insertion in a nucleic acid comprising 5-methylcytosine (5mC), 5-hydroxymethylcytosine (5hmC) or both, comprising
a nucleic acid comprising one or more 5-methylcytosine (5mC) or 5-hydroxymethylcytosine (5hmC);
a carbene precursor for producing a C—H insertion in the 5-methyl moiety of 5mC or the 5-hydroxymethyl moiety of 5hmC; and
a TET or a variant thereof;
wherein the carbene precursor is selected from
(a)
a structure of Formula I: 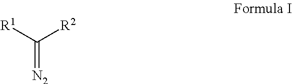
wherein
R¹ is selected from the group consisting of H, —C(O)OR^{1a}, C(O)R^{1a}, —C(O)N(R^{1b})₂, —SO₂R^{1a}, —SO₂OR¹, —P(O)(OR^{1a})₂, —NO₂, —CN, C₁₋₁₈ alkyl, C₂₋₁₈ alkenyl, C₂₋₁₈ alkynyl, 2- to 18-membered heteroalkyl, C₁₋₁₈ haloalkyl, C₁₋₁₈ alkoxy, C₃₋₁₀ cycloalkyl, C₆₋₁₀ aryl, 3- to 10-membered heterocyclyl, and 5- to 10-membered heteroaryl;
each R^{1a} is independently selected from the group consisting of H, C₁₋₁₈ alkyl, C₂₋₁₈ alkenyl, C₂₋₁₈ alkynyl, C₆₋₁₀ aryl, 3- to 10-membered heterocyclyl, and 5- to 10-membered heteroaryl;
each R^{1b} is independently selected from the group consisting of H, C₁₋₁₈ alkyl, C₂₋₁₈ alkenyl, C₁₋₁₈ alkynyl, and C₁₋₁₈ alkoxy;
R² is an electron-withdrawing group selected from the group consisting of —C(O)OR^{2a}, —C(O)R^{2a}, —C(O)N(R^{2b})₂, —SO₂R^{2a}, —SO₂OR^{2a}, —P(O)(OR²³)₂, —NO₂, and —CN;
each R^{2a} is independently selected from the group consisting of H, C₁₋₁₈ alkyl, C₂₋₁₈ alkenyl, C₂₋₁₈ alkynyl, C₆₋₁₀ aryl, 3- to 10-membered heterocyclyl, and 5- to 10-membered heteroaryl;
each R^{2b} is independently selected from the group consisting of H, C₁₋₁₈ alkyl, C₂₋₁₈ alkenyl, C₂₋₁₈ alkynyl, and C₁₋₈ alkoxy; and
R¹ and R² are optionally and independently substituted; or
R¹ and R² are taken together to form C₃₋₁₀ cycloalkyl, C₆₋₁₀ aryl, 3- to 10-membered heterocyclyl, and 5- to 10-membered heteroaryl, each of which is optionally substituted;
(b)
a structure of Formula I: 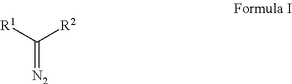
wherein
R¹ is selected from the group consisting of H, C(O)OR^{1a}, —C(O)R^{1a}, —C(O)N(R^{1b})₂, —SO₂R^{1a}, —SO₂OR^{1a}, —P(O)(OR^{1a})₂, —NO₂, —CN, C₁₋₁₈ alkyl, 2- to 18-membered heteroalkyl, C₁₋₁₈ haloalkyl, C₁₋₁₈ alkoxy, C₃₋₁₀ cycloalkyl, C₆₋₁₀ aryl, 3- to 10-membered heterocyclyl, and 5- to 10-membered heteroaryl;
each R^{1a} is independently C₁₋₈ alkyl;
each R^{1b} is independently selected from the group consisting of H, C₁₋₈ alkyl, and C₁₋₈ alkoxy;
R² is an electron-withdrawing group selected from the group consisting of —C(O)OR^{2a}, —C(O)R^{2a}, —C(O)N(R^{2b})₂, —SO₂R^{2a}, SO₂OR^{2a}, —P(O)(OR^{2a})₂, —NO₂, and —CN;
each R^{2a} is independently C₁₋₈ alkyl;
each R^{2b} is independently selected from the group consisting of H, C₁₋₈ alkyl, and C₁₋₈ alkoxy; and
R¹ and R² are optionally and independently substituted; or
R¹ and R² are taken together to form C₃₋₁₀ cycloalkyl, C₆₋₁₀ aryl, 3- to 10-membered heterocyclyl, and 5- to 10-membered heteroaryl, each of which is optionally substituted; or
(c) a structure of Formula I: 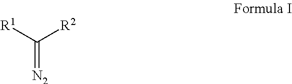
wherein
R¹ is independently selected from the group consisting of H, —C(O)OR^{1a}, —C(O)R^{1a}, —SO₂R^{1a}, —SO₂OR^{1a}, substituted C₁₋₁₈ alkyl, 2- to 18-membered heteroalkyl, C₁₋₁₈ alkoxy, C₃₋₁₀ cycloalkyl, C₁₋₁₈ fluoroalkyl, substituted C₆₋₁₀ aryl, and substituted 5- to 10-membered heteroaryl;
R^{1a} is C₁₋₈ alkyl;
R² is selected from the group consisting of —C(O)OR^{2a}, —C(O)R^{2a}, —SO₂R^{2a}, and —SO₂OR^{2a}; and
R^{2a} is C₁₋₈ alkyl; or
R¹ and R² are optionally taken together to form C₃₋₁₀ cycloalkyl, C₆₋₁₀ aryl, 3- to 10-membered heterocyclyl, and 5- to 10-membered heteroaryl, each of which is optionally substituted.

32-38. (canceled)

39. The reaction mixture of claim 31, wherein TET is selected from the group consisting of human TET1, TET2, TET3, and variants thereof; murine Tet1, Tet2, Tet3, and variants thereof; Naegleria TET (NgTET) and variants thereof; Coprinopsis cinerea (CcTET) and variants thereof, and a combination thereof.

40. The reaction mixture of claim 31, wherein the TET is TET1 or NgTET.

41-42. (canceled)

43. The reaction mixture of claim 31, comprising a non-reducing acid or a salt thereof.

44. (canceled)

45. The reaction mixture of claim 43, wherein the non-reducing acid is selected from the group consisting of acetic acid, dichloroacetic acid, fluoroacetic acid, chloroacetic acid, citric acid, ascorbic acid, benzoic acid, and a combination thereof.

46. The reaction mixture of claim 43, wherein the non-reducing acid is acetic acid or n-oxalylglycine.

47. The reaction mixture of claim 31, wherein the nucleic acid is DNA.

48. The reaction mixture of claim 31, wherein the nucleic acid is RNA.

49-50. (canceled)

51. The reaction mixture of claim 31,
wherein the reaction mixture does not comprise
(a) carboxy cytosine, dihydrouracil, uracil, or a combination thereof;
(b) cytidine deaminase;
(c) borane reagent; or
(d) combinations thereof.

52-55. (canceled)


SEQUENCE LISTING (7 sequences)

SEQ ID NO: 1 - protein, 2136 aa, Homo sapiens
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 2 - protein, 2002 aa, Homo sapiens
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 3 - protein, 1795 aa, Homo sapiens
MSQFQVPLAVQPDLPGLYDFPQRQVMVGSFPGSGLSMAGSESQLRGGGDGRKKRKRCGTCEPCRRLENCGACTSCTNRRTHQICKLRKCEVLKKKVGLLKEVEIKAGEGAGPWGQGAAVKTGSELSPVDGPVPGQMDSGPVYHGDSRQLSASGVPVNGAREPAGPSLLGTGGPWRVDQKPDWEAAPGPAHTARLEDAHDLVAFSAVAEAVSSYGALSTRLYETFNREMSREAGNNSRGPRPGPEGCSAGSEDLDTLQTALALARHGMKPPNCNCDGPECPDYLEWLEGKIKSVVMEGGEERPRLPGPLPPGEAGLPAPSTRPLLSSEVPQISPQEGLPLSQSALSIAKEKNISLQTAIAIEALTQLSSALPQPSHSTPQASCPLPEALSPPAPFRSPQSYLRAPSWPVVPPEEHSSFAPDSSAFPPATPRTEFPEAWGTDTPPATPRSSWPMPRPSPDPMAELEQLLGSASDYIQSVFKRPEALPTKPKVKVEAPSSSPAPAPSPVLQREAPTPSSEPDTHQKAQTALQQHLHHKRSLFLEQVHDTSFPAPSEPSAPGWWPPPSSPVPRLPDRPPKEKKKKLPTPAGGPVGTEKAAPGIKPSVRKPIQIKKSRPREAQPLFPPVRQIVLEGLRSPASQEVQAHPPAPLPASQGSAVPLPPEPSLALFAPSPSRDSLLPPTQEMRSPSPMTALQPGSTGPLPPADDKLEELIRQFEAEFGDSFGLPGPPSVPIQDPENQQTCLPAPESPFATRSPKQIKIESSGAVTVLSTTCFHSEEGGQEATPTKAENPLTPTLSGFLESPLKYLDTPTKSLLDTPAKRAQAEFPTCDCVEQIVEKDEGPYYTHLGSGPTVASIRELMEERYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLAGDNPKEEEVLRKSFQDLATEVAPLYKRLAPQAYQNQVTNEEIAIDCRLGLKEGRPFAGVTACMDFCAHAHKDQHNLYNGCTVVCTLTKEDNRCVGKIPEDEQLHVLPLYKMANTDEFGSEENQNAKVGSGAIQVLTAFPREVRRLPEPAKSCRQRQLEARKAAAEKKKIQKEKLSTPEKIKQEALELAGITSDPGLSLKGGLSQQGLKPSLKVEPQNHFSSFKYSGNAVVESYSVLGNCRPSDPYSMNSVYSYHSYYAQPSLTSVNGFHSKYALPSFSYYGFPSSNPVFPSQFLGPGAWGHSGSSGSFEKKPDLHALHNSLSPAYGGAEFAELPSQAVPTDAHHPTPHHQQPAYPGPKEYLLPKAPLLHSVSRDPSPFAQSSNCYNRSIKQEPVDPLTQAEPVPRDAGKMGKTPLSEVSQNGGPSHLWGQYSGGPSMSPKRTNGVGGSWGVFSSGESPAIVPDKLSSFGASCLAPSHFTDGQWGLFPGEGQQAASHSGGRLRGKPWSPCKFGNSTSALAGPSLTEKPWALGAGDFNSALKGSPGFQDKLWNPMKGEEGRIPAAGASQLDRAWQSFGLPLGSSEKLFGALKSEEKLWDPFSLEEGPAEEPPSKGAVKEEKGGGGAEEEEEELWSDSEHNFLDENIGGVAVAPAHGSILIECARRELHATTPLKKPNRCHPTRISLVFYQHKNLNQPNHGLALWEAKMKQLAERARARQEEAARLGLGQQEAKLYGKKRKWGGTVVAEPQQKEKKGVVPTRQALAVPTDSAVTVSSYAYTKVTGPYSRWI

SEQ ID NO: 4 - protein, 2007 aa, Mus musculus
MSRSRPAKPSKSVKTKLQKKKDIQMKTKTSKQAVRHGASAKAVNPGKPKQLIKRRDGKKETEDKTPTPAPSFLTRAGAARMNRDRNQVLFQNPDSLTCNGFTMALRRTSLSWRLSQRPVVTPKPKKVPPSKKQCTHNIQDEPGVKHSENDSVPSQHATVSPGTENGEQNRCLVEGESQEITQSCPVFEERIEDTQSCISASGNLEAEISWPLEGTHCEELLSHQTSDNECTSPQECAPLPQRSTSEVTSQKNTSNQLADLSSQVESIKLSDPSPNPTGSDHNGFPDSSFRIVPELDLKTCMPLDESVYPTALIRFILAGSQPDVFDTKPQEKTLITTPEQVGSHPNQVLDATSVLGQAFSTLPLQWGFSGANLVQVEALGKGSDSPEDLGAITMLNQQETVAMDMDRNATPDLPIFLPKPPNTVATYSSPLLGPEPHSSTSCGLEVQGATPILTLDSGHTPQLPPNPESSSVPLVIAANGTRAEKQFGTSLFPAVPQGFTVAAENEVQHAPLDLTQGSQAAPSKLEGEISRVSITGSADVKATAMSMPVTQASTSSPPCNSTPPMVERRKRKACGVCEPCQQKANCGECTYCKNRKNSHQICKKRKCEVLKKKPEATSQAQVTKENKRPQREKKPKVLKTDFNNKPVNGPKSESMDCSRRGHGEEEQRLDLITHPLENVRKNAGGMTGIEVEKWAPNKKSHLAEGQVKGSCDANLTGVENPQPSEDDKQQTNPSPTFAQTIRNGMKNVHCLPTDTHLPLNKLNHEEFSKALGNNSSKLLTDPSNCKDAMSVTTSGGECDHLKGPRNTLLFQKPGLNCRSGAEPTIFNNHPNTHSAGSRPHPPEKVPNKEPKDGSPVQPSLLSLMKDRRLTLEQVVAIEALTQLSEAPSESSSPSKPEKDEEAHQKTASLLNSCKAILHSVRKDLQDPNVQGKGLHHDTVVFNGQNRTFKSPDSFATNQALIKSQGYPSSPTAEKKGAAGGRAPFDGFENSHPLPIESHNLENCSQVLSCDQNLSSHDPSCQDAPYSQIEEDVAAQLTQLASTINHINAEVRNAESTPESLVAKNTKQKHSQEKRMVHQKPPSSTQTKPSVPSAKPKKAQKKARATPHANKRKKKPPARSSQENDQKKQEQLAIEYSKMHDIWMSSKFQRFGQSSPRSFPVLLRNIPVFNQILKPVTQSKTPSQHNELFPPINQIKFTRNPELAKEKVKVEPSDSLPTCQFKTESGGQTFAEPADNSQGQPMVSVNQEAHPLPQSPPSNQCANIMAGAAQTQFHLGAQENLVHQIPPPTLPGTSPDTLLPDPASILRKGKVLHFDGITVVTEKREAQTSSNGPLGPTTDSAQSEFKESIMDLLSKPAKNLIAGLKEQEAAPCDCDGGTQKEKGPYYTHLGAGPSVAAVRELMETRFGQKGKAIRIEKIVFTGKEGKSSQGCPVAKWVIRRSGPEEKLICLVRERVDHHCSTAVIVVLILLWEGIPRLMADRLYKELTENLRSYSGHPTDRRCTLNKKRTCTCQGIDPKTCGASFSFGCSWSMYFNGCKFGRSENPRKFRLAPNYPLHEKQLEKNLQELATVLAPLYKQMAPVAYQNQVEYEEVAGDCRLGNEEGRPFSGVTCCMDFCAHSHKDIHNMHNGSTVVCTLIRADGRDTNCPEDEQLHVLPLYRLADTDEFGSVEGMKAKIKSGAIQVNGPTRKRRLRFTEPVPRCGKRAKMKQNHNKSGSHNTKSFSSASSTSHLVKDESTDFCPLQASSAETSTCTYSKTASGGFAETSSILHCTMPSGAHSGANAAAGECTGTVQPAEVAAHPHQSLPTADSPVHAEPLTSPSEQLTSNQSNQQLPLLSNSQKLASCQVEDERHPEADEPQHPEDDNLPQLDEFWSDSEEIYADPSFGGVAIAPIHGSVLIECARKELHATTSLRSPKRGVPFRVSLVFYQHKSLNKPNHGFDINKIKCKCKKVTKKKPADRECPDVSPEANLSHQIPSRVASTLTRDNVVTVSPYSLTHVAGPYNRWV

SEQ ID NO: 5 - protein, 1912 aa, Mus musculus
MEQDRTTHAEGTRLSPFLIAPPSPISHTEPLAVKLQNGSPLAERPHPEVNGDTKWQSSQSCYGISHMKGSQSSHESPHEDRGYSRCLQNGGIKRTVSEPSLSGLHPNKILKLDQKAKGESNIFEESQERNHGKSSRQPNVSGLSDNGEPVTSTTQESSGADAFPTRNYNGVEIQVLNEQEGEKGRSVTLLKNKIVLMPNGATVSAHSEENTRGELLEKTQCYPDCVSIAVQSTASHVNTPSSQAAIELSHEIPQPSLTSAQINFSQTSSLQLPPEPAAMVTKACDADNASKPAIVPGTCPFQKAEHQQKSALDIGPSRAENKTIQGSMELFAEEYYPSSDRNLQASHGSSEQYSKQKETNGAYFRQSSKFPKDSISPTTVTPPSQSLLAPRLVLQPPLEGKGALNDVALEEHHDYPNRSNRTLLREGKIDHQPKTSSSQSLNPSVHTPNPPLMLPEQHQNDCGSPSPEKSRKMSEYLMYYLPNHGHSGGLQEHSQYLMGHREQEIPKDANGKQTQGSVQAAPGWIELKAPNLHEALHQTKRKDISLHSVLHSQTGPVNQMSSKQSTGNVNMPGGFQRLPYLQKTAQPEQKAQMYQVQVNQGPSPGMGDQHLQFQKALYQECIPRTDPSSEAHPQAPSVPQYHFQQRVNPSSDKHLSQQATETQRLSGFLQHTPQTQASQTPASQNSNFPQICQQQQQQQLQRKNKEQMPQTFSHLQGSNDKQREGSCFGQIKVEESFCVGNQYSKSSNFQTHNNTQGGLEQVQNINKNFPYSKILTPNSSNLQILPSNDTHPACEREQALHPVGSKTSNLQNMQYFPNNVTPNQDVHRCFQEQAQKPQQASSLQGLKDRSQGESPAPPAEAAQQRYLVHNEAKALPVPEQGGSQTQTPPQKDTQKHAALRWLLLQKQEQQQTQQSQPGHNQMLRPIKTEPVSKPSSYRYPLSPPQENMSSRIKQEISSPSRDNGQPKSIIETMEQHLKQFQLKSLCDYKALTLKSQKHVKVPTDIQAAESENHARAAEPQATKSTDCSVLDDVSESDTPGEQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKRSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTIYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 6 - protein, 1803 aa, Mus musculus
MSQFQVPLAVQPDLSGLYDFPQGQVMVGGFQGPGLPMAGSETQLRGGGDGRKKRKRCGTCDPCRRLENCGSCTSCTNRRTHQICKLRKCEVLKKKAGLLKEVEINAREGTGPWAQGATVKTGSELSPVDGPVPGQMDSGPVYHGDSRQLSTSGAPVNGAREPAGPGLLGAAGPWRVDQKPDWEAASGPTHAARLEDAHDLVAFSAVAEAVSSYGALSTRLYETFNREMSREAGSNGRGPRPESCSEGSEDLDTLQTALALARHGMKPPNCTCDGPECPDFLEWLEGKIKSMAMEGGQGRPRLPGALPPSEAGLPAPSTRPPLLSSEVPQVPPLEGLPLSQSALSIAKEKNISLQTAIAIEALTQLSSALPQPSHSTSQASCPLPEALSPSAPFRSPQSYLRAPSWPVVPPEEHPSFAPDSPAFPPATPRPEFSEAWGTDTPPATPRNSWPVPRPSPDPMAELEQLLGSASDYIQSVFKRPEALPTKPKVKVEAPSSSPAPVPSPISQREAPLLSSEPDTHQKAQTALQQHLHHKRNLFLEQAQDASFPTSTEPQAPGWWAPPGSPAPRPPDKPPKEKKKKPPTPAGGPVGAEKTTPGIKTSVRKPIQIKKSRSRDMQPLFLPVRQIVLEGLKPQASEGQAPLPAQLSVPPPASQGAASQSCATPLTPEPSLALFAPSPSGDSLLPPTQEMRSPSPMVALQSGSTGGPLPPADDKLEELIRQFEAEFGDSFGLPGPPSVPIQEPENQSTCLPAPESPFATRSPKKIKIESSGAVTVLSTTCFHSEEGGQEATPTKAENPLTPTLSGFLESPLKYLDTPTKSLLDTPAKKAQSEFPTCDCVEQIVEKDEGPYYTHLGSGPTVASIRELMEDRYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLTGDNPKEEEVLRNSFQDLATEVAPLYKRLAPQAYQNQVTNEDVAIDCRLGLKEGRPFSGVTACMDFCAHAHKDQHNLYNGCTVVCTLTKEDNRCVGQIPEDEQLHVLPLYKMASTDEFGSEENQNAKVSSGAIQVLTAFPREVRRLPEPAKSCRQRQLEARKAAAEKKKLQKEKLSTPEKIKQEALELAGVTTDPGLSLKGGLSQQSLKPSLKVEPQNHFSSFKYSGNAVVESYSVLGSCRPSDPYSMSSVYSYHSRYAQPGLASVNGFHSKYTLPSFGYYGFPSSNPVFPSQFLGPSAWGHGGSGGSFEKKPDLHALHNSLNPAYGGAEFAELPGQAVATDNHHPIPHHQQPAYPGPKEYLLPKVPQLHPASRDPSPFAQSSSCYNRSIKQEPIDPLTQAESIPRDSAKMSRTPLPEASQNGGPSHLWGQYSGGPSMSPKRTNSVGGNWGVFPPGESPTIVPDKLNSFGASCLTPSHFPESQWGLFTGEGQQSAPHAGARLRGKPWSPCKFGNGTSALTGPSLTEKPWGMGTGDFNPALKGGPGFQDKLWNPVKVEEGRIPTPGANPLDKAWQAFGMPLSSNEKLFGALKSEEKLWDPFSLEEGTAEEPPSKGVVKEEKSGPTVEEDEEELWSDSEHNFLDENIGGVAVAPAHCSILIECARRELHATTPLKKPNRCHPTRISLVFYQHKNLNQPNHGLALWEAKMKQLAERARQRQEEAARLGLGQQEAKLYGKKRKWGGAMVAEPQHKEKKGAIPTRQALAMPTDSAVTVSSYAYTKVTGPYSRWI

SEQ ID NO: 7 - protein, 321 aa, Naegleria gruberi
MTTFKQQTIKEKETKRKYCIKGTTANLTQTHPNGPVCVNRGEEVANTTTLLDSGGGINKKSLLQNLLSKCKTTFQQSFTNANITLKDEKWLKNVRTAYFVCDHDGSVELAYLPNVLPKELVEEFTEKFESIQTGRKKDTGYSGILDNSMPFNYVTADLSQELGQYLSEIVNPQINYYISKLLTCVSSRTINYLVSLNDSYYALNNCLYPSTAFNSLKPSNDGHRIRKPHKDNLDITPSSLFYFGNFQNTEGYLELTDKNCKVFVQPGDVLFFKGNEYKHVVANITSGWRIGLVYFAHKGSKTKPYYEDTQKNSLKIHKETK